SEQUENCE LISTING (58 sequences)

SEQ ID NO: 1 - DNA, 20 bp, Artificial Sequence
  Synthesized
gtgcagtgccgatatacctc

SEQ ID NO: 2 - DNA, 20 bp, Artificial Sequence
  Synthesized
agatattcctcatactggaa

SEQ ID NO: 3 - DNA, 25 bp, Artificial Sequence
  Synthesized
aggatgtcgagggatggcaggcagc

SEQ ID NO: 4 - DNA, 26 bp, Artificial Sequence
  Synthesized
tgccagcatcgctcatcctcagatcc

SEQ ID NO: 5 - DNA, 25 bp, Artificial Sequence
  Synthesized
caccgagatattcctcatactggaa

SEQ ID NO: 6 - DNA, 25 bp, Artificial Sequence
  Synthesized
aaacttccagtatgaggaatatctc

SEQ ID NO: 7 - protein, 20 aa, Mus musculus
QEQGSLTVQCRYTSGWKDYK

SEQ ID NO: 8 - DNA, 60 bp, Mus musculus
caggagcagggctccttgacagtgcagtgccgatatacctcaggctggaaggattacaag

SEQ ID NO: 9 - DNA, 58 bp, Artificial Sequence
  Synthesized
caggagcagggctccttgacagtgcagtgccgatatctcaggctggaaggattacaag

SEQ ID NO: 10 - DNA, 59 bp, Artificial Sequence
  Synthesized
caggagcagggctccttgacagtgcagtgccgatatactcaggctggaaggattacaag

SEQ ID NO: 11 - DNA, 13 bp, Artificial Sequence
  Synthesized
caggagcagggag

SEQ ID NO: 12 - DNA, 53 bp, Artificial Sequence
  Synthesized
caggagcagggctccttgacagtgcagtgccctcaggctggaaggattacaag

SEQ ID NO: 13 - DNA, 59 bp, Artificial Sequence; identical to SEQ ID NO: 10
  Synthesized

SEQ ID NO: 14 - protein, 20 aa, Mus musculus
EQGSLTVQCRYSSYWKGYKK

SEQ ID NO: 15 - DNA, 60 bp, Mus musculus
gagcagggctccttgacagtgcagtgcagatattcctcatactggaagggttacaagaag

SEQ ID NO: 16 - DNA, 52 bp, Artificial Sequence
  Synthesized
gagcagggctccttgacagtgcagtgcagatattcctcagggttacaagaag

SEQ ID NO: 17 - DNA, 59 bp, Artificial Sequence
  Synthesized
gagcagggctccttgacagtgcagtgcagatattcctcatactgaagggttacaagaag

SEQ ID NO: 18 - DNA, 59 bp, Artificial Sequence; identical to SEQ ID NO: 17
  Synthesized

SEQ ID NO: 19 - DNA, 59 bp, Artificial Sequence
  Synthesized
gagcagggctccttgacagtgcagtgcagatattcctcatacggaagggttacaagaag

SEQ ID NO: 20 - DNA, 20 bp, Artificial Sequence
  Synthesized
cacgccaccgatctgttctg

SEQ ID NO: 21 - DNA, 17 bp, Artificial Sequence
  Synthesized
gcgctgcgccatcactc

SEQ ID NO: 22 - protein, 4 aa, Mus musculus
KDYK

SEQ ID NO: 23 - protein, 4 aa, Homo sapiens
ETYL

SEQ ID NO: 24 - protein, 4 aa, Mus musculus
RDFI

SEQ ID NO: 25 - protein, 4 aa, Homo sapiens
KNRT

SEQ ID NO: 26 - protein, 10 aa, Mus musculus
TKGGLDPMFK

SEQ ID NO: 27 - protein, 10 aa, Homo sapiens
EKTGNDLGVT

SEQ ID NO: 28 - DNA, 20 bp, Artificial Sequence
  Synthesized
tgtcactgtgaagatggtgg

SEQ ID NO: 29 - DNA, 20 bp, Artificial Sequence
  Synthesized
acaggtccagaggaggtgag

SEQ ID NO: 30 - DNA, 20 bp, Artificial Sequence
  Synthesized
ggatccagtcacaggtccag

SEQ ID NO: 31 - DNA, 20 bp, Artificial Sequence
  Synthesized
gttactgtgaacattggccc

SEQ ID NO: 32 - DNA, 20 bp, Artificial Sequence
  Synthesized
ctagctactactctgataac

SEQ ID NO: 33 - DNA, 20 bp, Artificial Sequence
  Synthesized
cctgtgatccagaagagaaa

SEQ ID NO: 34 - DNA, 20 bp, Artificial Sequence
  Synthesized
ggtcgtccctgatggacaca

SEQ ID NO: 35 - DNA, 20 bp, Artificial Sequence
  Synthesized
ctccacgtaggtacaccgca

SEQ ID NO: 36 - DNA, 20 bp, Artificial Sequence
  Synthesized
cgtagccagacacagagcca

SEQ ID NO: 37 - DNA, 20 bp, Artificial Sequence
  Synthesized
tcagagaaagaaacgaagag

SEQ ID NO: 38 - DNA, 20 bp, Artificial Sequence
  Synthesized
gcccttgctacttacctggg

SEQ ID NO: 39 - DNA, 20 bp, Artificial Sequence
  Synthesized
gcagcaaatccaaatctcag

SEQ ID NO: 40 - DNA, 20 bp, Artificial Sequence
  Synthesized
gctacttacctgggaggaaa

SEQ ID NO: 41 - DNA, 20 bp, Artificial Sequence
  Synthesized
gagtgtgtcatgtcgatatg

SEQ ID NO: 42 - DNA, 20 bp, Artificial Sequence
  Synthesized
taccacgccacaggactcca

SEQ ID NO: 43 - DNA, 20 bp, Artificial Sequence
  Synthesized
ggaagaggatgagcagagca

SEQ ID NO: 44 - DNA, 20 bp, Artificial Sequence
  Synthesized
ctacgcaaatcaggaccagg

SEQ ID NO: 45 - DNA, 20 bp, Artificial Sequence
  Synthesized
acggaagtgagaaagaaaag

SEQ ID NO: 46 - DNA, 20 bp, Artificial Sequence
  Synthesized
tagagcagagaactgccaca

SEQ ID NO: 47 - DNA, 20 bp, Artificial Sequence
  Synthesized
cataggtcactgtaaaggtg

SEQ ID NO: 48 - protein, 110 aa, Mus musculus
EDPVTGPEEVSGQEQGSLTVQCRYTSGWKDYKKYWCQGVPQRSCKTLVETDASEQLVKKNRVSIRDNQRDFIFTVTMEDLRMSDAGIYWCGITKGGLDPMFKVTVNIGPA

SEQ ID NO: 49 - protein, 110 aa, Mus musculus
QDSVTGPEEVSGQEQGSLTVQCRYSSYWKGYKKYWCRGVPQRSCDILVETDKSEQLVKKNRVSIRDNQRDFIFTVTMEDLRMSDAGIYWCGITKGGPDPMFKVNVNIDQA

SEQ ID NO: 50 - protein, 110 aa, Mus musculus
QDPVTGPEEVSGQEQGSLTVQCRYDSGWKDYKKYWCRGAYWKSCEILVETDASEQLVKENRVSIRDDQTDFIFTVTMEDLRMSDADIYWCGITKAGTDPMFKVNVNIDPE

SEQ ID NO: 51 - protein, 107 aa, Mus musculus
IQGPALVRGPEQGSVTVQCRYSSRWQTNKKWWCRGASWSTCRVLIRSTGSEKETKSGRLSIRDNQKNHSFQVTMEMLRQNDTDTYWCGIEKFGTDRGTRVKVNVYSV

SEQ ID NO: 52 - protein, 113 aa, Mus musculus
LTGPGSVSGYVGGSLRVQCQYSPSYKGYMKYWCRGPDHTTCKTIVETDGSEKEKRSGPVSIRDHASNSTITVIMEDLSEDNAGSYWCKIQTSFIWDSWSRDPSVSVRVNVFPA

SEQ ID NO: 53 - protein, 116 aa, Mus musculus
HFPVRGPSTVTGTVGESLSVSCQYEKKLKTKKKIWCKWKSNVLCKDIVKTSASEEARNRGVSIRDHPDNLTFTVTLENLTLEDDAGTYMCMDIGFFYDAYLQIDKSFKVEVFVVPG

SEQ ID NO: 54 - protein, 112 aa, Mus musculus
LHGPSTMSGSVGESLSVSCRYEEKFKTKDKYWCRVSLKILCKDIVKTSSSEEARSGRVTIRDHPDNLTFTVTYESLTLEDADTYMCAVDISLFDGSLGFDKYFKIELSVVPS

SEQ ID NO: 55 - protein, 108 aa, Mus musculus
LKGPKEISGFEGDTVSLRCTYVEKMKEHRKYWCRQGGILVSRCGDIVYANQDQEVTRGRMSIRDSPQELSMTVIMRDLTLKDSGKYWCGIDRLGRDESFEVTLIVFPG

SEQ ID NO: 56 - protein, 108 aa, Homo sapiens
QITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPA

SEQ ID NO: 57 - protein, 337 aa, Mus musculus
MHLSLLVPFLFWITGCCTAEDPVTGPEEVSGQEQGSLTVQCRYTSGWKDYKKYWCQGVPQRSCKTLVETDASEQLVKKNRVSIRDNQRDFIFTVTMEDLRMSDAGIYWCGITKGGLDPMFKVTVNIGPAIQVPITVPTMPPITSTTTIFTVTTTVKETSMFPTLTSYYSDNGHGGGDSGGGEDGVGDGFLDLSVLLPVISAVLLLLLLVASLFAWRMVRRQKKAAGPPSEQAQSLEGDLCYADLSLKQPRTSPGSSWKKGSSMSSSGKDHQEEVEYVTMAPFPREEVSYAALTLAGLGQEPTYGNTGCPITHVPRTGLEEETTEYSSIRRPLPAAMP

SEQ ID NO: 58 - protein, 221 aa, Mus musculus
MWQFSALLLFFLPGCCTAQDSVTGPEEVSGQEQGSLTVQCRYSSYWKGYKKYWCRGVPQRSCDILVETDKSEQLVKKNRVSIRDNQRDFIFTVTMEDLRMSDAGIYWCGITKGGPDPMFKVNVNIDQAPKSSMMTTTATVLKSIQPSAENTGKEQVTQSKEVTQSRPHTRSLLSSIYFLLMVFVELPLLLSMLSAVLWVTRPQRCFGRGENDLVKTHSPVA

(19) United States
(12) Patent Application Publication
(10) Pub. No.: US 2018/0252712 A1
(43) Pub. Date: Sep. 6, 2018

(54) RECEPTOR FOR NOROVIRUS AND USES THEREOF

(71) Applicants: Herbert Virgin, St. Louis, MO (US); Daved Fremont, St. Louis, MO (US); Craig Wilen, St. Louis, MO (US); Robert Orchard, St. Louis, MO (US); Christopher Nelson, St. Louis, MO (US)

(72) Inventors: Herbert Virgin, St. Louis, MO (US); Daved Fremont, St. Louis, MO (US); Craig Wilen, St. Louis, MO (US); Robert Orchard, St. Louis, MO (US); Christopher Nelson, St. Louis, MO (US)

(73) Assignee: Washington University, St. Louis, MO (US)

(21) Appl. No.: 15/909,594

(22) Filed: Mar. 1, 2018

Related U.S. Application Data
(60) Provisional application No. 62/465,604, filed on Mar. 1, 2017.

(51) Int. Cl. G01N 33/569; C12N 7/02

(52) U.S. Cl. CPC G01N 33/56983; C12N 7/02; G01N 2500/10; G01N 2333/08; G01N 2800/52; C12N 2503/02; C12N 2320/10; G01N 2500/04

(57) ABSTRACT

The present disclosure provides compositions and cell comprising a norovirus receptor as well as methods of use thereof. Such uses include identification of anti-viral compounds, treatment of norovirus infection, and development of an in vitro culture system for norovirus.

### CROSS REFERENCE TO RELATED APPLICATIONS

This application claims the benefit of U.S. provisional application No. 62/465,604, filed on Mar. 1, 2017, the disclosure of which is hereby incorporated by reference in its entirety.

### GOVERNMENTAL RIGHTS

This invention was made with government support under A1109725 awarded by National Institutes of Health. The government has certain rights in the invention.

### FIELD OF THE INVENTION

The present disclosure provides compositions and cells comprising a norovirus receptor as well as methods of use thereof. Such uses include identification of anti-viral compounds, treatment of norovirus infection, and development of an in vitro culture system for norovirus.

### BACKGROUND OF THE INVENTION

Noroviruses (NoV) are non-enveloped positive-sense RNA viruses and represent a leading cause of gastroenteritis in humans worldwide. Due to the strict species tropism of viruses in the NoV genus and the lack of robust replication of human noroviruses (HNoV) in cell culture and animal models, murine norovirus (MNoV) has emerged as a model system to uncover basic mechanisms of NoV biology in vitro and in vivo. MNoVs can establish persistent enteric infection enabling studies of the interplay between viral persistence, resident enteric microorganisms, and the host immune system. Importantly, the capacity of HNoV and MNoV to bind cells, and the susceptibility of humans to HNoV infection, have been linked to expression of cell surface and secreted carbohydrates, while other members of the Caliciviridae utilize proteinaceous receptors. Despite the importance of this viral genus, host factors including receptor(s) required for NoV infection and pathogenesis have largely defied molecular identification; their discovery would aid in understanding mechanisms of NoV replication, vaccination, species tropism and enteric viral persistence. Thus, there is a need in the art to identify host molecules required for MNoV infection and HNoV infection.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C and FIG. 1D depict a schematic, heatmap and graphs showing that a CRISPR screen identifies CD300lf as necessary for MNoV infection. (FIG. 1A) Mouse BV2 cells stably expressing Cas9 were transduced with lentivirus encoding sgRNAs with genome-wide coverage in each of four unique pools. sgRNAs from cells surviving MNoV challenge were analyzed. (FIG. 1B) A heat map showing enrichment of genes in the four indicated conditions. Genes are color coded based upon their STARS score. (FIG. 1C, FIG. 1D) Wild-type BV2 cells or two independently derived CD300lf-deficient clones (clone 1 and clone 2) transduced with an empty vector or a vector expressing CD300lf were challenged with an MOI of 0.05 of (FIG. 1C) MNoV^{CW3} or (FIG. 1D) MNoV^{CR6} infection and virus production was assessed by plaque forming units (PFU). L.O.D., limit of detection.

FIG. 2A, FIG. 2B, FIG. 2C, FIG. 2D, FIG. 2E, FIG. 2F, FIG. 2G, FIG. 2H and FIG. 2I depict graphs showing that CD300lf is an MNoV receptor. (FIG. 2A) Indicated cells were transfected with MNoV^{CW3} RNA and harvested 12 hours post transfection. Viral production was measured by plaque assay. ns=not statistically significant. (FIG. 2B) BV2 cells were incubated with either α-CD300lf or an isotype control prior to infection with MNoV^{CW3} at an MOI 5.0. Cell viability was measured 24 hours post-infection. (FIG. 2C) MNoV^{CW3} was incubated with either recombinant, soluble ectodomain of CD300lf (sCD300lf) or a control protein prior to infection of BV2 cells. Cellular viability was assayed 24 hours post-infection. (FIG. 2D) MNoV^{CW3} pretreated with indicated proteins prior to infection of bone marrow-derived macrophages (BMDM) for 16 hours. Infection was measured by FACS for intracellular MNoV NS1/2 expression. (FIG. 2E) BMDM were preincubated with incubated with either α-CD300lf or a control antibody prior to infection with MNoV^{CW3}. Infection was measured by FACS for intracellular MNoV NS1/2 expression. (FIG. 2F) MNoV^{CW3} harvested from the spleens of BL6/J mice or MNoV^{CW3} derived from BV2 cells was preincubated with sCD300lf prior to plaque assay. (FIG. 2G, FIG. 2H) Survival of STAT1−/− mice from challenge with (FIG. 2G) 10³ PFU or (FIG. 2H) 10⁵ PFU of MNoV^{CW3} preincubated with either sCD300lf or control protein. (FIG. 2I) MNoV^{CW3} binding assay to indicated BV2 cell lines as assayed by bound MNoV genomes via qPCR. Representative binding assay when cells or virus were preincubated with α-CD300lf or sCD300lf, respectively.

FIG. 3A, FIG. 3B, FIG. 3C, FIG. 3D, FIG. 3E and FIG. 3F depict graphs and images showing that structural analysis indicates the CDReq3 loop of CD300lf as critical for MNoV infection. (FIG. 3A, FIG. 3B). HeLa cells transiently transfected with given construct were infected with either (FIG. 3A) MNoV^{CW3} or (FIG. 3B) MNoV^{CR6} at an MOI of 0.05. Viral growth was measured by plaque assay at the indicated time points. (FIG. 3C) HeLa cells transiently transfected with indicated CD300 constructs were infected with MNoV^{CW3} at an MOI of 5.0 and analyzed for expression of CD300 (FLAG) and MNoV NS1/2 expression. (FIG. 3D) Ribbon diagram of CD300lf with bound metal ion and HEPES. The β-sheets in cyan are lettered as a canonical V-type Ig domain with the positions of the CDR equivalent loops indicated. The disulfide bonds are shown in yellow. (FIG. 3E) HeLa cells transiently transfected with indicated CD300 constructs were infected with MNoV^{CW3} at an MOI of 5.0 and analyzed for expression of CD300 (FLAG) and MNoV NS1/2 expression. CD300lfΔCT, CD300lf with truncation of cytoplasmic domain; CD300lfCDReq3, CD300lf with human CDReq3 sequence. (FIG. 3F) Mapping of mutations results onto the CD300lf surface. Displayed in cyan are murine CD300lf residues replaced by human equivalent residues in CDR1 (SEQ ID NO: 22-23) (²⁹KDYK³²→ETYL), ED loop (SEQ ID NO: 24-25) (⁶⁹RDFI⁷²→KNRT), and the tip of CDR3 (⁹⁵GGL⁹⁷→AGT) that had no effect on infection, along with mutation of the CD300-family conserved D⁹⁸ into A. The side chains altered by each mutation are shown as ball-and-stick figures beneath a transparent surface. The complete CDR3 mutation that disrupted viral infection is shown in magenta (a swap of (SEQ ID NO: 26) murine ⁹³TKGGLDPMFK¹⁰² with (SEQ ID NO: 27) human EKTGNDLGVT).

FIG. 4A and FIG. 4B depict sgRNA validation of screening hits in BV2-Cas9 cells. Survival of BV2-Cas9 cells with sgRNA targeting indicated genes two days post-infection with MNoV^{CW3} (top) and MNoV^{CR6} (bottom) at an MOI of 0.05. Each gene was targeted with four unique sgRNA constructs, and the dashed line indicates the relative viability of cells with a transduction control.

FIG. 5A, FIG. 5B and FIG. 5C depict MNoV binding occurs independently of carbohydrates on host cells. (FIG. 5A) MNoV^{CW3} genome copies in indicated tissues of Fut2 wild-type (WT) or knockout (KO) mice seven days post infection. (FIG. 5B) FACS plots of fluorescein isothiocyonate- conjugated wheat germ agglutinin (WGA-FITC) binding to BV2 cells treated with kifunensine. (FIG. 5C) A representative MNoV^{CW3} binding assay with kifunensine treated or untreated cells. The mean fluorescence intensity (MFI) of WGA-FITC binding of cells is displayed below each bar to indicate depletion of complex cell surface carbohydrates. ***p<0.0001.

FIG. 6A and FIG. 6B depict transiently CD300 is detectable on HeLa cells used for MNoV. (FIG. 6A) Expression of CD300-FLAG was confirmed by Western blot prior to MNoV infection. (FIG. 6B) Susceptibility of CD300 expressing HeLa cells challenged with MNoV^{CW3} (MOI 5) was measured by co-staining for intracellular MNoV NS1/2 and CD300-Flag by FACS.

FIG. 7A and FIG. 7B depict CD300ld is not required for MNoV infection of BV2 cells. Relative viability of indicated BV2 cells one day post infection with (FIG. 7A) MNoV^{CW3} and (FIG. 7B) MNoV^{CR6} at an MOI 5.0 or 0.05.

FIG. 8 depicts refined 2Fo-Fc electron density map from the 1.6 Å resolution murine sCD300lf data set. The map is contoured at 1.8a, where a represents the root mean square of the density fluctuation of the unit cell, and centered around the metal ion. The model structure is shown as ball-and-sticks. The carbon atoms are colored cyan for sCD300lf and green for HEPES. Nitrogen atoms are colored blue, oxygens red and sulphurs yellow.

FIG. 9 depicts structure-based sequence alignment of the extracellular Ig V-type domains of the CD300 family. Structure-based sequence alignment of the extracellular Ig V-type domains of the CD300 family. Complete sequence conservation is represented with white text on a red background. In this alignment, the secondary-structures of murine CD300lf are shown by arrows above the sequences and the secondary-structures of human CD300lf (RCSB entry 2NMS) are shown below. The positions of the CDRs are marked as equivalent to an immunoglobulin V-domain. At the bottom is a heat map of the relative solvent accessibility of murine CD300lf residues with red being most solvent accessible and blue being least accessible. Starred residues are in contact with the coordinated metal ion and HEPES. The numbering corresponds to the mature (signal-cleaved) form of murine CD300lf. (SEQ ID NOs: 48-56).

FIG. 10 depicts structural comparison of murine and human CD300lf. The coordinates of the human CD300lf Ig domain (RCSB entry 2NMS; yellow) have been superimposed onto the murine structure (purple) with an overall rmsd of 1.07 Å for 107 aligned Cα-positions. The greatest structural variation between the two structures is readily apparent in the CC′-loop (residues 38-44; rmsd of 2.41 Å) and CDR3 (residues 93-99; rmsd of 2.18 Å).

### DETAILED DESCRIPTION OF THE INVENTION

Noroviruses are a leading cause of gastroenteritis globally but host factors required for norovirus (NoV) infection are poorly understood. The inventors identified host molecules essential for murine NoV (MNoV) induced cell death including CD300lf as a proteinaceous receptor. CD300lf is essential for MNoV binding and replication in both cell lines and primary cells. The soluble ectodomain of CD300lf protects against lethal MNoV infection in vivo. Expression of CD300lf in human cells breaks the species barrier restricting MNoV replication. The crystal structure of the CD300lf ectodomain revealed a potential ligand binding cleft composed of residues critical for MNoV infection. Therefore, the presence of a proteinaceous receptor is the primary determinant of MNoV species tropism while other components of cellular machinery required for NoV replication are conserved between humans and mice.

In an aspect, the disclosure provides an immortalized human cell, wherein the cell expresses at least a portion of murine CD300lf or CD300ld. The immortalized human cell is susceptible to infection by murine norovirus (MNoV).

In another aspect, the disclosure provides a method of identifying a compound that inhibits infection of MNoV. The method comprises: (a) contacting a compound and MNoV with an immortalized human cell, wherein the cell expresses at least a portion of murine CD300lf or CD300ld; and (b) determining MNoV titers, wherein an amount of MNoV that is reduced relative to control indicates inhibition of infection of MNoV. The control is not contacted with the compound.

In another aspect, the disclosure provides a method of identifying proteins and/or nucleic acids involved in norovirus infection. The method comprises: (a) contacting MNoV with an immortalized human cell, wherein the cell expresses at least a portion of murine CD300lf or CD300ld; and (b) evaluating protein and/or nucleic acid expression relative to an immortalized human cell expressing murine CD300lf not contacted with MNoV. In a different aspect, the disclosure provides a method of identifying proteins and/or nucleic acids involved in norovirus infection. The method comprises: (a) genetically modulating a host cell nucleic acid or protein expression in an immortalized human cell, wherein the cell expresses at least a portion of murine CD300lf or CD300ld; (b) contacting MNoV with the immortalized cell and (b) determining MNoV titers or infectivity relative to an immortalized human cell expressing at least a portion of murine CD300lf or CD300ld which has not been genetically modulated.

In still another aspect, the disclosure provides a method of screening for antiviral compounds to treat human norovirus (HNoV) infection. The method comprises: (a) contacting a compound and MNoV with an immortalized human cell, wherein the cell expresses at least a portion of murine CD300lf or CD300ld; and (b) determining MNoV titers, wherein an amount of MNoV that is reduced relative to control indicates an antiviral compound that may treat HNoV.

In a different aspect, the disclosure provides an isolated amino acid sequence comprising at least a portion of murine CD300lf protein. In another aspect, the disclosure provides an isolated amino acid sequence comprising at least a portion of murine CD300ld protein. These amino acid sequences may be capable of protecting against a norovirus infection. In another aspect, the present disclosure provides a nucleic acid molecule which encodes an amino acid comprising at least a portion of a CD300lf of CD300ld protein.

In another aspect, the disclosure provides a method to treat a MNoV infection, the method comprising administering a composition comprising at least a portion of murine CD300lf or CD300ld. In still another aspect, the disclosure provides a method to treat or prevent a MNoV infection, the method comprising administering a composition comprising an antibody directed to CD300lf or CD300ld.

### I. Compositions

In an aspect, the present disclosure provides for isolated polypeptides comprising at least a portion of CD300. These polypeptides may be capable of specifically forming a complex with a norovirus. In addition, the polypeptide may be soluble in an aqueous solution. In one embodiment, the polypeptides comprise murine CD300lf or fragments thereof. In a specific embodiment, the polypeptides comprise the ectodomain of murine CD300lf. In another embodiment the polypeptides comprise CD300ld or fragments thereof. In still another embodiment, the polypeptides comprise the ectodomain of CD300ld. The soluble amino acid sequence of the present invention may be used as a therapeutic agent, i.e. a prophylactic, for the treatment of a subject infected with a norovirus. Moreover, a monoclonal antibody directed to the soluble amino acid sequence of the present invention may be useful as a vaccine for immunizing a subject against a norovirus.

As used herein the term “CD300” refers to a family of cell surface molecules that modulate a diverse array of cell processes via their paired triggering and inhibitory receptor functions. Non-limiting examples include CD300lf, CD300ld, CD300lh, CD300lb, CD300e, CD300c, CD300a, CD300lg as well as all orthologs thereof. In preferred embodiments, the polypeptides of the invention comprise a soluble fragment of CD300lf or a soluble fragment of CD300ld. In certain embodiments, the polypeptide of the invention comprise the ectodomain of CD300lf. Unless otherwise indicated, “polypeptide” shall include a protein, protein domain, or peptide, and any fragment thereof. The murine CD300lf polypeptide has the amino acid sequence as set forth in UniProtKB accession no. Q6SJQ7, herein incorporated by reference. The murine CD300lf polypeptide has the amino acid sequence as set forth in UniProtKB accession no. Q8VCH2, herein incorporated by reference. CD300lf and CD300ld contain four distinct domains structures which include an N-terminal signal domain, an ectodomain, a transmembrane domain, and a C-terminal cytoplasmic domain. The ectodomain is the region of the polypeptide presented in the extracelluar space. The ectodomains of CD300lf and CD300ld are highly homologous; the 112 aa distal to the signal-peptide sequence encompass the N-terminal region and showed 85.7% homology between the two molecules. The two motifs in the N-terminal region of CD300lf, from P22 to C55 and from E68 to K113, were 94.1% and 93.3% identical to those in CD300ld, respectively.

Homologs can be identified by comparison of amino acid sequence, e.g., manually or by using known homology-based search algorithms such as those commonly known and referred to as BLAST, FASTA, and Smith-Waterman. A local sequence alignment program, e.g., BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails search of the significant hits against a database of amino acid sequences from the base organism that are similar to the sequence of the query protein. A hit is a likely ortholog, when the reciprocal query's best hit is the query protein itself or a protein encoded by a duplicated gene after speciation. In some embodiments, the CD300 polypeptide is a human CD300 polypeptide or a mouse CD300 polypeptide.

In one embodiment, a CD300 polypeptide of the disclosure may comprise an amino acid sequence with 80, 81, 82, 83, 84, 85, 86, 87, 88, 89, 90, 91, 92, 93, 94, 95, 96, 97, 98, 99, or 100% identity to SEQ ID NO:57. In one embodiment, a CD300 polypeptide of the disclosure may comprise an amino acid sequence with 80, 81, 82, 83, 84, 85, 86, 87, 88, 89, 90, 91, 92, 93, 94, 95, 96, 97, 98, 99, or 100% identity to SEQ ID NO:58. In one embodiment, a CD300 polypeptide of the disclosure may comprise an amino acid sequence with 80, 81, 82, 83, 84, 85, 86, 87, 88, 89, 90, 91, 92, 93, 94, 95, 96, 97, 98, 99, or 100% identity to SEQ ID NO:48. In one embodiment, a CD300 polypeptide of the disclosure may comprise an amino acid sequence with 80, 81, 82, 83, 84, 85, 86, 87, 88, 89, 90, 91, 92, 93, 94, 95, 96, 97, 98, 99, or 100% identity to SEQ ID NO:49.

Another aspect of the present disclosure provides nucleic acids encoding any of the CD300 molecules described above. The nucleic acid can be DNA or RNA. In one embodiment the DNA can be present in a vector. The nucleic acid sequences which encode the dominant negative molecule of the invention can be operatively linked to expression control sequences. “Operatively linked” refers to a juxtaposition wherein the components so described are in a relationship permitting them to function in their intended manner. An expression control sequence operatively linked to a coding sequence is achieved under conditions compatible with the expression control sequences. As used herein, the expression control sequences refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, and maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

The present disclosure provides for a vector comprising a CD300 molecule. The vector can be a plasmid, cosmid, yeast artificial chromosome (YAC), bacterial artificial chromosome (BAC), viral vector or bacteriophage. The vectors can provide for replication of CD300 nucleic acids, expression of CD300 polypeptides or integration of CD300 nucleic acids into the chromosome of a host cell. The choice of vector is dependent on the desired purpose. Certain cloning vectors are useful for cloning, mutation and manipulation of the CD300 nucleic acid. Other vectors are useful for expression of the CD300 polypeptide, being able to express the polypeptide in large amounts for purification purposes. The vector can also be chosen on the basis of the host cell, e.g., to facilitate expression in bacteria, mammalian cells, insect cells, fish cell (e.g., zebrafish) and/or amphibian cells. The choice of matching vector to host cell is apparent to one of skill in the art, and the types of host cells are discussed below. Many vectors or vector systems are available commercially, for example, the pET bacterial expression system (Invitrogen™, Carlsbad Calif.).

The vectors disclosed herein can be viral or non-viral vectors. For example, the disclosed vectors can be viral vectors. Specifically, the disclosed vectors can be adenoviral vectors. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain neurodegenerative diseases or disorders and cell populations by using the targeting characteristics of the carrier.

Vectors can include various components including, but not limited to, an origin of replication, one or more marker or selectable genes (e.g. GFP, neo), promoters, enhancers, terminators, poly-adenylation sequences, repressors or activators. Such elements are provided in the vector so as to be operably linked to the coding region of the CD300-encoding nucleic acid, thereby facilitating expression in a host cell of interest. Cloning and expression vectors can contain an origin of replication which allows the vector to replicate in the host cells. Vectors can also include a selectable marker, e.g., to confer a resistance to a drug or compliment deficiencies in growth. Examples of drug resistance markers include, but are not limited to, ampicillin, tetracycline, neomycin or methotrexate. Examples of other marker genes can be the fluorescent polypeptides such one of the members of the fluorescent family of proteins, for example, GFP, YFP, BFP, RFP etc. These markers can be contained on the same vector as the gene of interest or can be on separate vectors and co-transfected with the vector containing the gene of interest.

The vector can contain a promoter that is suitable for expression of CD300 in mammalian cells, which promoter can be operably linked to provide for inducible or constitutive expression of CD300. Exemplary inducible promoters include, for example, the metallothionine promoter or an ecdysone-responsive promoter. Exemplary constitutive promoters include, for example, the viral promoters from cytomegalovirus (CMV), Rous Sarcoma virus (RSV), Simian virus 40 (SV40), avian sarcoma virus, the beta-actin promoter and the heat-shock promoters.

The vector encoding CD300 molecule can be a viral vector. Examples of viral vectors include retroviral vectors, such as: adenovirus, simian virus 40 (SV40), cytomegalovirus (CMV), Moloney murine leukemia virus (MoMuLv), Rous Sarcoma Virus (RSV), lentivirus, herpesvirus, poxvirus and vaccinia virus. A viral vector can be used to facilitate expression in a target cell, e.g., for production of CD300 or for use in therapy (e.g., to deliver the CD300 polypeptide to a patient by expression from the vector). Where used for therapy, CD300-encoding vectors (e.g, viral vectors), can be administered directly to the patient via an appropriate route or can be administered using an ex vivo strategy using patient cells (autologous) or allogeneic cells, which are suitable for administration to the patient to be treated.

The present disclosure provides a cell culture system wherein mammalian cells are susceptible to infection by murine norovirus. Development of the compositions and methods of the invention involved the discovery that expression of at least a portion of CD300lf or CD300ld polypeptide in a previously non-permissive cell renders the cell susceptible to a norovirus infection. The term “heterologous expression”, as used herein, shall refer to the translation of a gene, a nucleic acid or a cDNA, which is non-native to the organism in which the expression occurs. As used herein, a “norovirus-permissive cell” is a cell in which a norovirus replicates following infection with a norovirus or transfection with norovirus genome RNA. As used herein, “norovirus replication” can be understood to include various stages in norovirus life cycle, such as, for example, binding of a norovirus to a host cell, entry into the host cell, trafficking, processing, genome release, translation, transcription, assembly, and release. In some embodiments, norovirus replication can be detected by measuring norovirus protein activity, for example polyprotein protease activity, viral RNA polymerase activity, VPG activity or NTPase activity. In some configurations, measurement of an increased accumulation of viral RNA or viral protein in infected cells can be considered an indication of viral replication, although an increase in virus particle production is not measured. Hence, in certain configurations, in a test of a candidate anti-viral agent, anti-viral activity can be detected by detecting inhibition of viral nucleic acid synthesis, or by detecting inhibition of a norovirus protein activity, such as inhibition of polyprotein protease activity, viral RNA polymerase activity, VPG activity or NTPase activity. Furthermore, in certain configurations, in a test of a candidate anti-viral agent, anti-viral activity can be detected by detecting inhibition of formation, disassembly or degradation of a viral RNA replicative intermediate such as a viral lariat structure. In other configurations, in a test of a candidate anti-viral agent, anti-viral activity can be detected by detecting inhibition of a norovirus protein accumulation, such as inhibition of polyprotein protease accumulation, viral RNA polymerase accumulation, VPG accumulation or NTPase accumulation. In one embodiment, the mammalian cell is an immortalized human cell. Examples of mammalian cells include, but are not limited to, macrophage-lineage cells, dendritic cell-lineage cells, Chinese hamster ovary (CHO) cells, HEK 293 cells, human cervical carcinoma cells (Hela), canine kidney cells (MDCK), human liver cells (HepG2), baby hamster kidney cells (BHK), and monkey kidney cells (CV1). In one embodiment, the mammalian cells are primary cells.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro. The term “immortalized cell” or “immortal cell” refers to any cells that are not limited by the Hayflick limit. The term “Hayflick limit,” as used herein, is defined as the number of times that differentiated cells can divide (e.g., about 50 times) before dying. As cells approach this limit, they show signs of aging. A primary cell culture is a culture from a cell or taken directly from a living organism, which is not immortalized.

As used herein, the term “norovirus” can refer to unmodified, wild-type norovirus, e.g., norovirus obtained from an individual with viral gastroenteritis, unless specified otherwise. As used herein, the term “host range-modified norovirus” refers to norovirus modified, with regard to its host range, using laboratory methods, e.g., norovirus grown in vitro for multiple passages.

A composition of the disclosure may optionally comprise one or more additional drugs or therapeutically active agents in addition to the CD300 molecules of the invention. In some embodiments, a composition of the disclosure may further comprise a pharmaceutically acceptable excipient. Non-limiting examples of suitable pharmaceutically acceptable excipients include a diluent, a binder, a filler, a buffering agent, a pH modifying agent, a disintegrant, a dispersant, a preservative, a lubricant, taste-masking agent, a flavoring agent, a coloring agent, or a combination thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate. examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In general, such compositions include a CD300 molecule and a suitable pharmaceutically acceptable carrier. The phrase “pharmaceutically acceptable carrier” refers to compositions that facilitate the delivery of a CD300 molecule including, but not limited to, solvents or dispersants, coatings, isotonic agents, agents that mediate absorption time or release of a CD300 molecule, and the like. Formulations suitable for bolus delivery of a CD300 molecule are contemplated, as are sustained release formulations to provide depot injections (e.g., implants).

Non-limiting examples of pharmaceutically acceptable carriers, include physiological saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, wool fat or a combination thereof.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride in the composition.

Prolonged absorption of the injectable compositions can be brought about by including in the composition an agent which delays absorption, for example, aluminum monostearate and gelatin.

The methods for preparing pharmaceutical compositions of the invention will be known to those skilled in the art and are described in Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve a desired effect in a subject that can be described as a clinical benefit in a CD300-based therapy-responsive patient. A dosage of the CD300 molecule can be about 0.1 to 100 mg/kg of body weight per day. If the CD300 molecule is administered to a body cavity or into an organ, the dose range can be adjusted lower or higher depending on the response.

The CD300 molecule of the invention can be administered by a medically acceptable route. Administration routes include injection, for example the injection can be subcutaneous, intraocular, intravenous, intramuscular or intravascular etc. Administration can include inhalation, oral, ocular, nasal, rectal, or ophthalmic routes.

An effective amount of the CD300 molecule that prevents or slows the progression of norovirus infection is known as a “prophylactic effective dose.” A prophylactic effective dose will depend on the factors of weight, age, administration route, and seriousness of the predisposition.

As used herein, the terms “pharmaceutically effective” or “therapeutically effective” shall mean an amount of a composition comprising a CD300 molecule of the invention that is sufficient to show a meaningful patient benefit, i.e., treatment, prevention, amelioration, or decrease in the frequency of the condition or symptom being treated.

### II. Methods

In one aspect the invention provides a method of producing norovirus-permissive cells. Specifically, the method includes heterologous expression of CD300lf or CD300ld in cells previously non-permissive to a MNoV infection. In various embodiments, the present invention can involve methods of replicating a norovirus in vitro. The norovirus-permissive culture and the accompanying methods can be used for a variety of purposes, such as diagnostic methods, development of assays for viral replication, selection of mutant viruses with desirable properties, identification of mutant viruses, screening of potential anti-viral compounds, and development of vaccines.

In various embodiments, the invention comprises methods of identifying a compound having anti-viral activity. “Anti-viral activity,” as used herein, can comprise inhibiting viral activity at any stage in a virus' life cycle. Hence, anti-viral activity can comprise, by way of non-limiting example, inhibition of viral replication, inhibition of viral gene expression, or inhibition of a viral protein accumulation or activity. Inhibition of a viral protein accumulation or activity can comprise, by way of non-limiting example, inhibition of norovirus polyprotein protease accumulation, inhibition of norovirus RNA polymerase accumulation, inhibition of norovirus VPG accumulation, inhibition of norovirus NTPase accumulation, inhibition of norovirus polyprotein protease activity, inhibition of norovirus RNA polymerase activity, inhibition of norovirus VPG activity inhibition of norovirus NTPase activity, inhibition of lariat formation, or inhibition of lariat degradation. Standard methods well known in the art for measuring or detecting norovirus protein accumulation or activity can be used, for example, enzyme assays and antibody assays.

In certain configurations, a method for identifying a compound having anti-viral activity can comprise contacting a candidate anti-viral compound with a norovirus-permissive cell culture of the invention infected with a norovirus, and detecting inhibition of norovirus replication. In certain aspects, a candidate anti-viral compound can be added to an infected norovirus-permissive culture at a concentration of from about 1 picomolar to about 100 millimolar, or from about 1 nanomolar to about 100 micromolar. Detecting inhibition of viral replication in some embodiments can thus comprise detecting inhibition of viral nucleic acid synthesis or viral protein synthesis. In some configurations, detecting inhibition of norovirus replication can comprise performing a plaque assay on the norovirus-permissive cell culture. A plaque assay can comprise determining a titer of virus accumulated in a plaque formed by infected cells in the presence of the candidate anti-viral molecule. In these configurations, assays for identifying anti-viral compounds can be used for identifying compounds having anti-RNA virus activity, anti-single-stranded RNA virus activity, anti-positive strand single-stranded RNA virus activity, anti-positive strand single-stranded RNA, no DNA stage virus activity, anti-calicivirus activity, or anti-norovirus activity. A norovirus infecting a norovirus-permissive cell by these methods can be, in certain configurations, a norovirus comprising a nucleic acid consisting of from about 7200 to about 7700 nucleotides. In some configurations, anti-viral activity can be detected by detecting differences between infected norovirus-permissive cells contacted with a candidate anti-viral agent and control infected norovirus-permissive cells. Such differences can comprise, by way of non-limiting example, gene expression differences, antigenic differences, enzyme activity differences, dye-staining differences, or morphological differences (as revealed by light microscopy or electron microscopy). In some configurations, anti-viral activity can be detected by performing a cytopathic effects (CPE) inhibition assay in which the anti-viral activity reduces or prevents norovirus-induced CPE.

In various embodiments, the invention comprises methods of identifying proteins and/or nucleic acids involved in norovirus infection. The proteins or nucleic acids may be important at any stage in a virus' life cycle. In non-limiting examples the proteins or nucleic acids may be important for viral replication, viral gene expression, or viral protein accumulation or activity. A genome wide genetic screen as described herein can be utilized in the methods of the invention. In non-limiting examples, a large-scale RNAi-based screen or CRISPR-based screen may be used to identify nucleic acids or proteins which modulate the infectivity of a norovirus. In one aspect the method includes contacting MNoV with a norovirus permissive cell of the invention and evaluating how the protein and/or nucleic acid expression relates to MNoV infectivity relative to a control norovirus permissive cell where the host cells nucleic acid or protein expression is unchanged. In one aspect, an exogenous gene or protein of interest introduced into a MNoV permissive cell of the invention.

The term “exogenous” and exogenous gene”, as used herein refers to nucleic acid sequences which are introduced to and/or expressed within a target cell. The exogenous nucleic acid sequences may be intact (that is, full-length sequences) or may be cleaved within the cell at one or more cleavage sites.

As referred to herein, the terms “gene of interest” and “exogenous gene of interest”, may interchangeably be used. The terms refer to a nucleic acid sequence which may encode for any structural or functional molecule subsequently expressed in the target cell.

As referred to herein, the terms “protein of interest” and “exogenous protein of interest”, may interchangeably be used. The terms refer to a peptide sequence which is translated from an exogenous RNA molecule, within a cell.

In various embodiments, the invention provides methods to treat or prevent a norovirus infection, the methods comprising administering to a subject a therapeutically effective amount of a composition comprising CD300 molecules as disclosed herein, for instance those described in Section I.

Suitable subjects include, but are not limited to, a human, a livestock animal, a companion animal, a lab animal, and a zoological animal. A subject may or may not be known to have a norovirus infection. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the animal is a laboratory animal. Non-limiting examples of a laboratory animal may include rodents, canines, felines, and non-human primates. In another preferred embodiment, the subject is a human.

Generally speaking, a therapeutically effective amount of a composition comprising a CD300 molecule is administered to a subject. In specific embodiments, the composition comprises a soluble ectodomain of CD300lf or CD300ld. Actual dosage levels of active ingredients in a therapeutic composition of the disclosure may be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, viral symptoms, and the physical condition and prior medical history of the subject being treated. In some embodiments, a minimal dose is administered, and the dose is escalated in the absence of dose-limiting toxicity.

Determination and adjustment of a therapeutically effective dose, as well as evaluation of when and how to make such adjustments, are known to those of ordinary skill in the art of medicine. In certain embodiments, the dose may range from 0.01 g to 10 g. For example, the dose may range from about 0.1 g to about 5 g, or from about 0.5 g to about 5 g, or from about 1 g to about 10 g, or from about 1 g to about 5 g. Additionally, the dose may be about 0.01 g, about 0.05 g, about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, about 4 g, about 4.1 g, about 4.2 g, about 4.3 g, about 4.4 g, about 4.5 g, about 4.6 g, about 4.7 g, about 4.8 g, about 4.9 g, about 5 g, about 6 g, about 6.5 g, about 7 g, about 7.5 g, about 8 g, about 8.5 g, about 9 g, about 9.5 g, about 10 g, or more than 10 g.

The frequency of dosing may be once, twice or three times or more daily or once, twice, three times or more per week or per month, as needed as to effectively treat the symptoms. For example, the frequency of dosing may be once, twice, or three times daily for one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months or more. Additionally, the frequency of dosing may be once daily for one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 2 years, 3 years, 4 years, 5 years, or more than 5 years. In certain embodiments, the duration of treatment could range from a single dose administered on a one-time basis to a life-long course of therapeutic treatments.

The timing of administration of the treatment relative to the infection itself and duration of treatment will be determined by the circumstances surrounding the case. Treatment could begin immediately after exposure to a virus, or exposure to an environment where the virus is common. Treatment may begin in a hospital or clinic, or at a later time after discharge from the hospital or after being seen in an outpatient clinic

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. CD300lf is a Receptor for Murine Norovirus

Noroviruses (NoV) are non-enveloped positive-sense RNA viruses that represent a leading cause of gastroenteritis in humans worldwide [1, 2]. Due to the strict species tropism of viruses in the NoV genus and the lack of robust replication of human noroviruses (HNoV) in cell culture and animal models, murine norovirus (MNoV) has emerged as a model system to uncover basic mechanisms of NoV biology in vitro and in vivo [3-9]. MNoVs can establish persistent enteric infection enabling studies of the interplay between viral persistence, resident enteric microorganisms, and the host immune system [4, 6, 7]. Importantly, the capacity of HNoV and MNoV to bind to cells, and the susceptibility of humans to HNoV infection, have been linked to expression of cell surface and secreted carbohydrates [5, 10-12], while other members of the Caliciviridae utilize proteinaceous receptors [13, 14]. Despite the importance of this viral genus, host factors including receptor(s) required for NoV infection and pathogenesis have largely defied molecular identification; their discovery would aid in understanding mechanisms of NoV replication, vaccination, species tropism and enteric viral persistence. To identify host molecules required for MNoV infection we undertook an unbiased forward genetic approach.

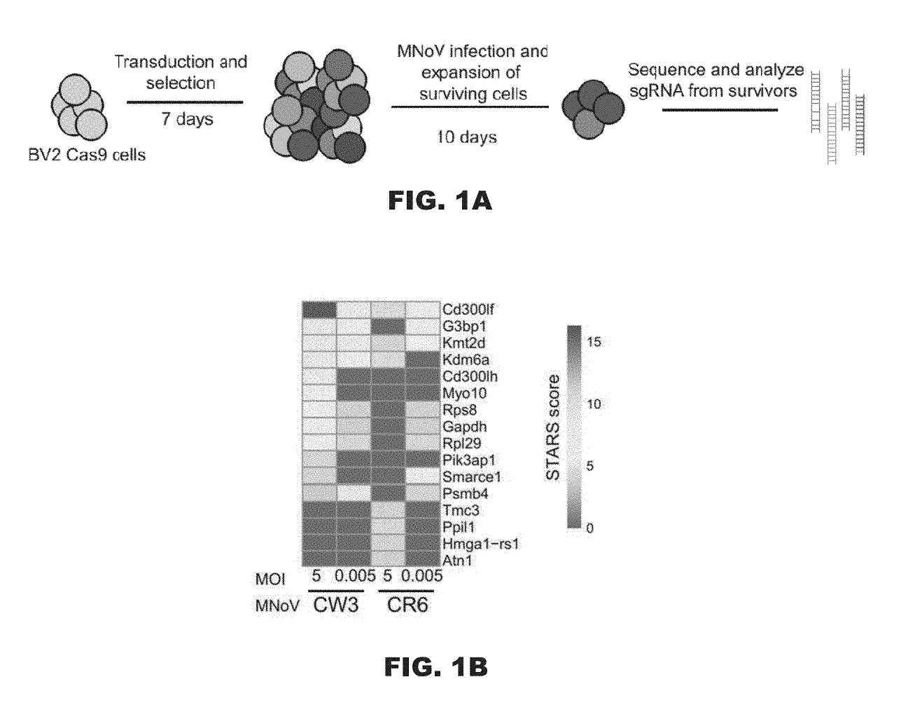

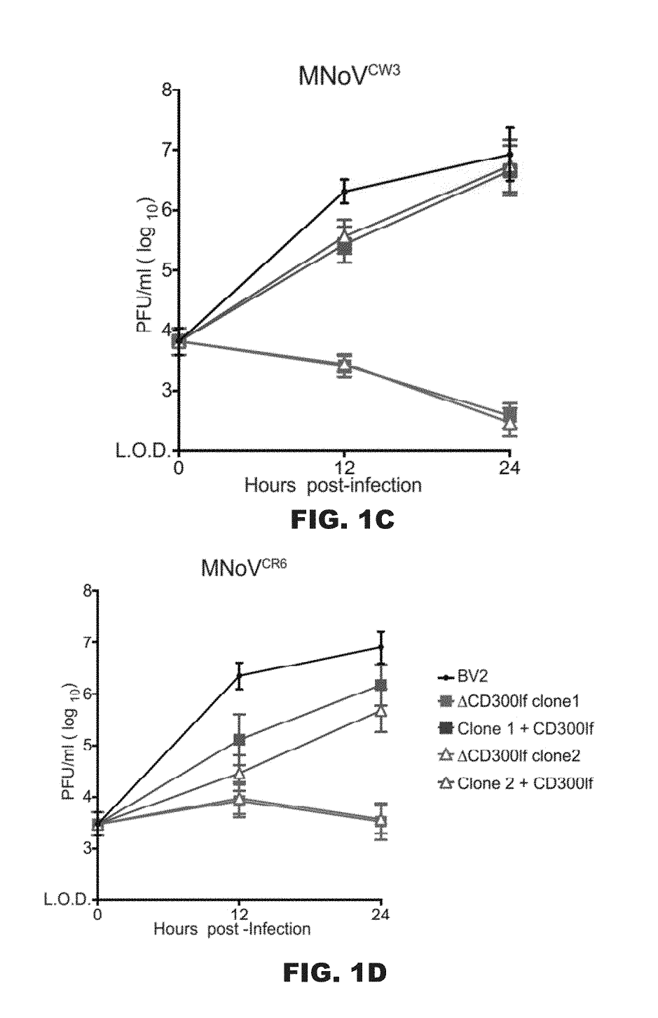

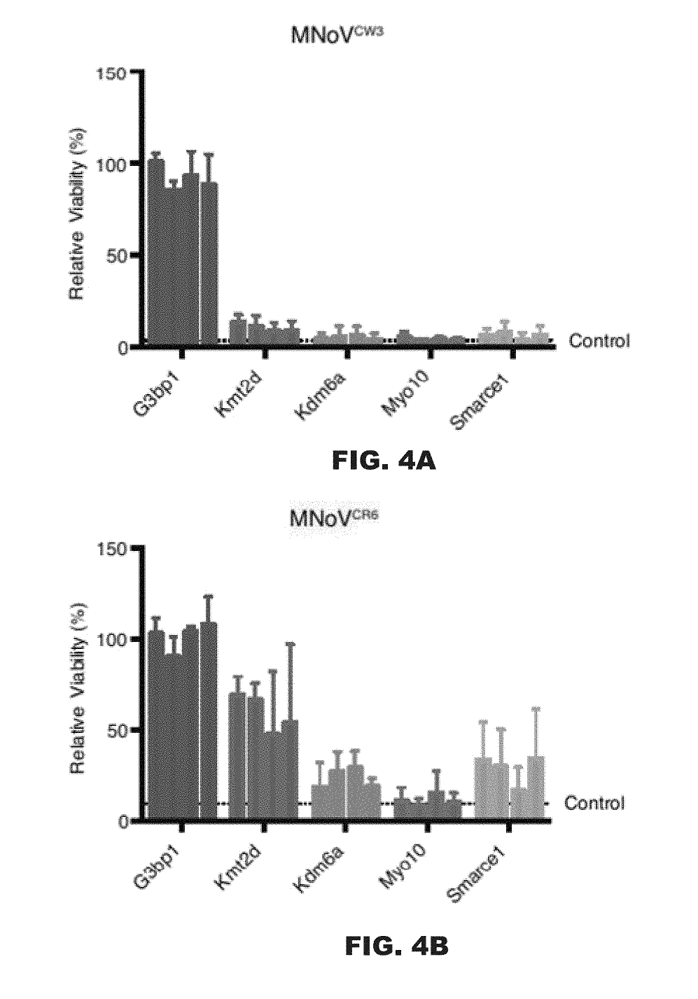

MNoV replicates and induces cell death in murine macrophage-like cells, including the microglial BV2 cell line, allowing identification of genes essential for MNoV replication using CRISPR-Cas9 technology. We introduced four independent genome-wide subpools of single guide RNAs (sgRNAs) of the murine Asiago library into BV2 cells stably expressing Cas9, and then infected the cells with MNoV strains that either cause acute systemic infection (MNoV^{CW3}) or persistent enteric infection (MNoV^{CR6}) in mice [15, 16]. sgRNA sequences from the surviving cells were sequenced and analyzed using the STARS gene-ranking algorithm (FIG. 1A) [16]. sgRNAs targeting CD300lf (also known as CLM-1, CMRF35, MAIR-V, LMIR3, IgSF13, IREM1, Digr2, and Pigr3), a cell surface immunoglobulin (Ig) domain-containing molecule within a family of proteins involved in binding lipids, were most abundantly enriched for both MNoV strains (FIG. 1B, and Table 1-5) [17]. We generated two independent BV2ΔCD300lf clones; the growth of MNoV^{CW3} and MNoV^{CR6} was abolished in both clones. Complementation with CD300lf cDNA restored MNoV replication in both clones, confirming the on-target activity of the sgRNAs (FIG. 1C, FIG. 1D). Several additional molecules predicted to be in the cytoplasm or nucleus were identified (FIG. 1B). Individual sgRNAs targeting five of these genes were introduced into BV2-Cas9 cells and four of the five genes were determined to be necessary for MoNV induced cell death, validating the utility of our screen to identify essential host factors for NoV (FIG. 4). Taken together, these data provide a systematic overview of the molecules required for NoV replication.

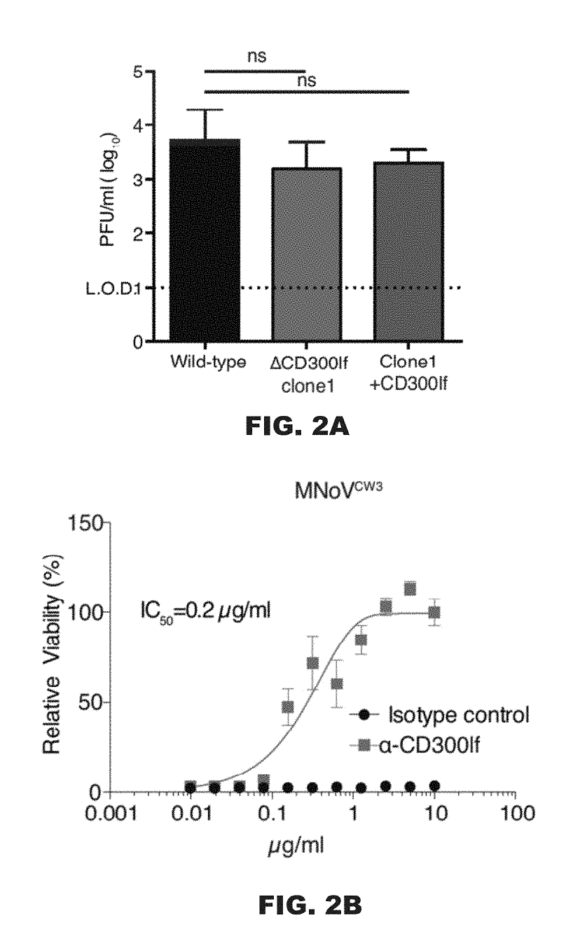

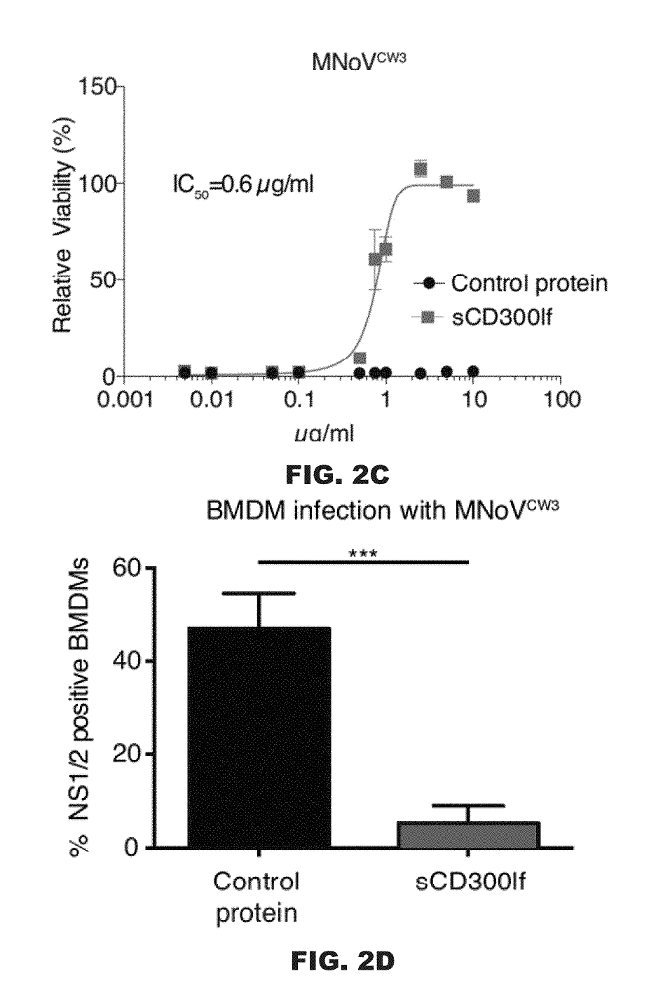

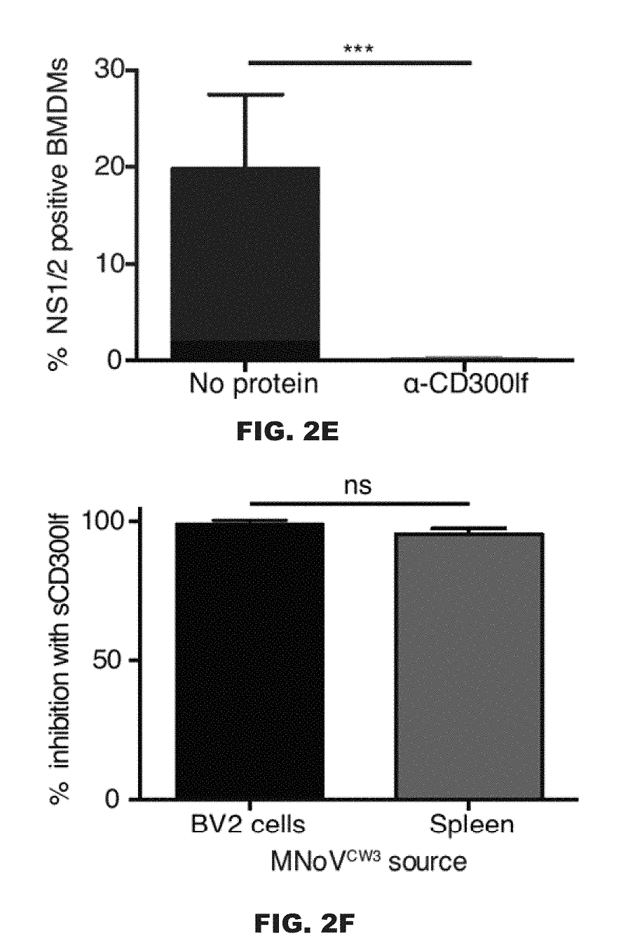

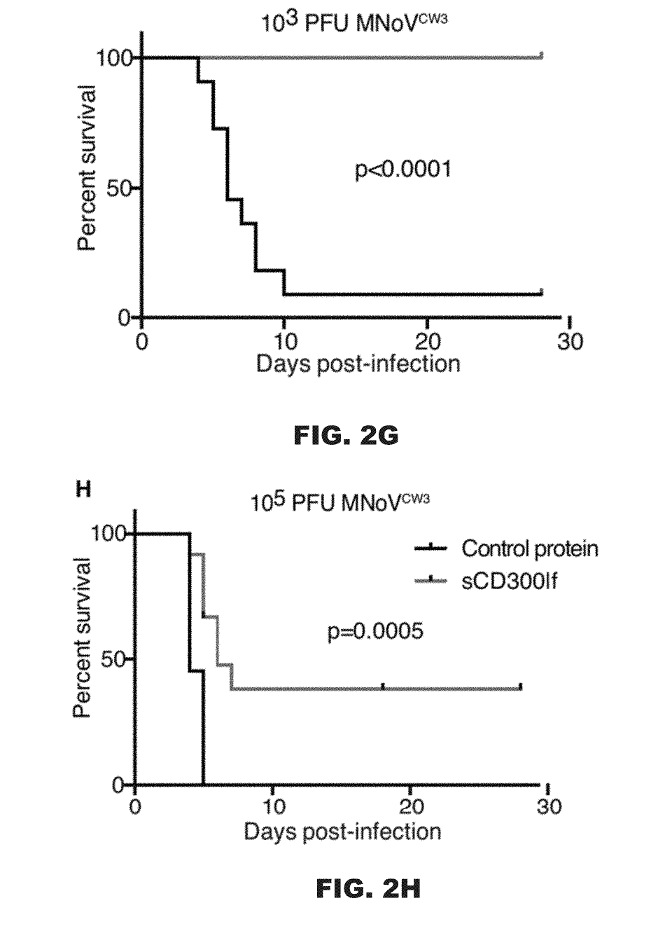

We selected CD300lf for further analysis because of its cell surface expression and the importance of viral receptors for conferring permissiveness for viral replication, species tropism, and pathogenesis. Transfection of MNoV^{CW3} RNA into BV2ΔCD300lf cells was sufficient to rescue MNoV^{CW3} production at wild-type levels, demonstrating that CD300lf is essential for viral entry, perhaps as a viral receptor (FIG. 2A). Consistent with this hypothesis, preincubation of cells with a polyclonal antibody targeting CD300lf (α-CD300lf) blocked MNoV^{CW3}-induced cytopathic effect in BV2 cells (FIG. 2B). Similarly, incubating MNoV^{CW3} with recombinant CD300lf ectodomain (sCD300lf) neutralized MNoV^{CW3}-induced cytopathic effect (FIG. 2C). RNA viruses are well known to adapt to use non-physiologic receptors during passage in cultured cells. However, MNoV^{CW3} infection of primary bone marrow-derived macrophages (BMDMs) was inhibited by preincubating the virus with sCD300lf or treating cells with α-CD300lf (FIG. 2D, FIG. 2E). Furthermore, MNoV isolated from the spleens of MNoV^{CW3}-infected mice remained sensitive to sCD300lf inhibition, indicating that sCD300lf neutralizes MNoV regardless of the source of the virus (FIG. 2F). Finally, to test the role of CD300lf and MNoV interactions in vivo, we incubated MNoV^{CW3} with either sCD300lf or a control protein prior to infection of STAT1^{-/-} mice. In a dose-dependent manner, sCD300lf preincubation was able to protect from MNoV induced lethality (FIG. 2G, FIG. 2H). Taken together these data indicate that the interactions between MNoV and CD300lf are essential for MNoV infection in vitro and in vivo.

To directly test if CD300lf functions as a binding receptor for MNoV, we analyzed the attachment of MNoV^{CW3} to BV2 cells. Mutation of CD300lf significantly reduced MNoV^{CW3} binding to cells, and binding was restored upon expression of CD300lf cDNA (FIG. 2I). Additionally, treating BV2 cells with α-CD300lf, or preincubating virus with sCD300lf, reduced MNoV binding (FIG. 2I). α-CD300lf treatment inhibited binding to wild type cells more robustly than did mixing virus with sCD300lf, potentially owing to differences in bivalent binding of antibody to CD300lf on the cell surface compared to binding of a monomeric Ig domain to the virus. Importantly, in BV2ΔCD300lf cells, binding of MNoV^{CW3} was not further inhibited with α-CD300lf or by premixing the virus with sCD300lf (FIG. 2I). Taken together, these data indicate CD300lf mediates viral binding and is a functional receptor for MNoV.

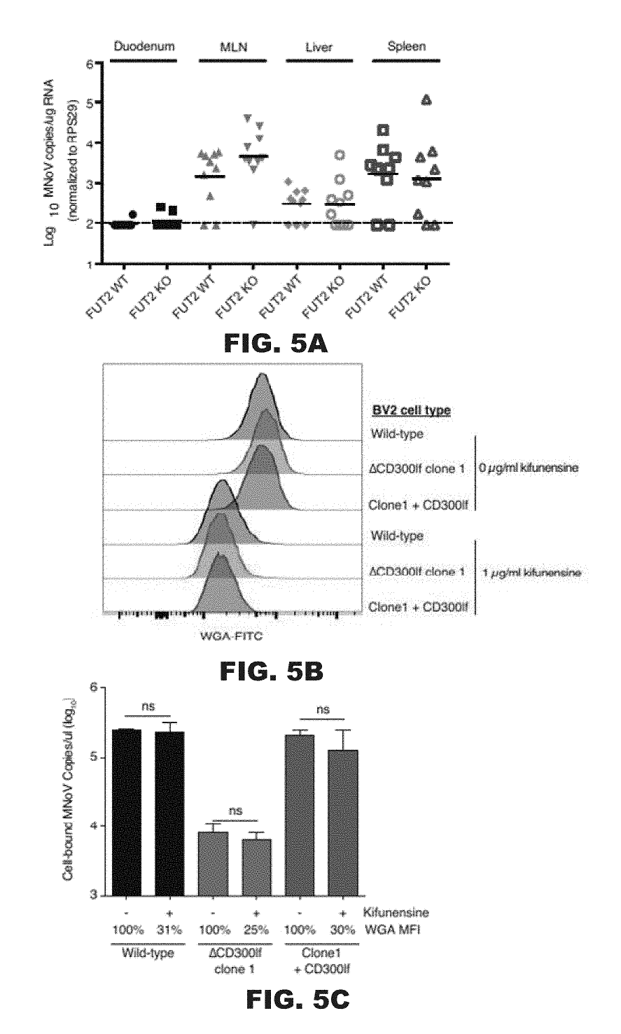

Previous reports have suggested that carbohydrates facilitate the binding of MNoV and HNoV to host cells and control the susceptibility of humans to HNoV infection [5, 10-12]. Therefore, we assessed the relative contribution of carbohydrates to MNoV attachment and infection. Surprisingly, mice deficient in Fut2, which controls histo-blood group antigen (HBGA) secretor status, had similar viral loads compared to wild-type, littermate controls infected with MNoV^{CW3} (FIG. 5). Also, treating cells with the mannosidase I inhibitor kifunensine significantly reduced cell surface carbohydrates, but did not significantly alter MNoV^{CW3} binding to either wild type or CD300lf-mutant cells (FIG. 5). These data suggest that MNoV binding and infection is dependent upon CD300lf, but not on carbohydrates within the sensitivity of these assays.

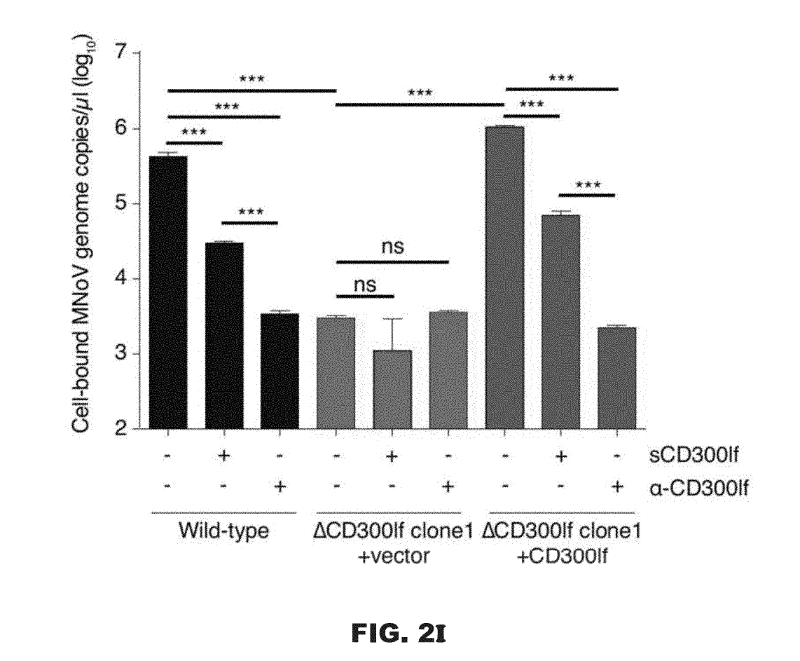

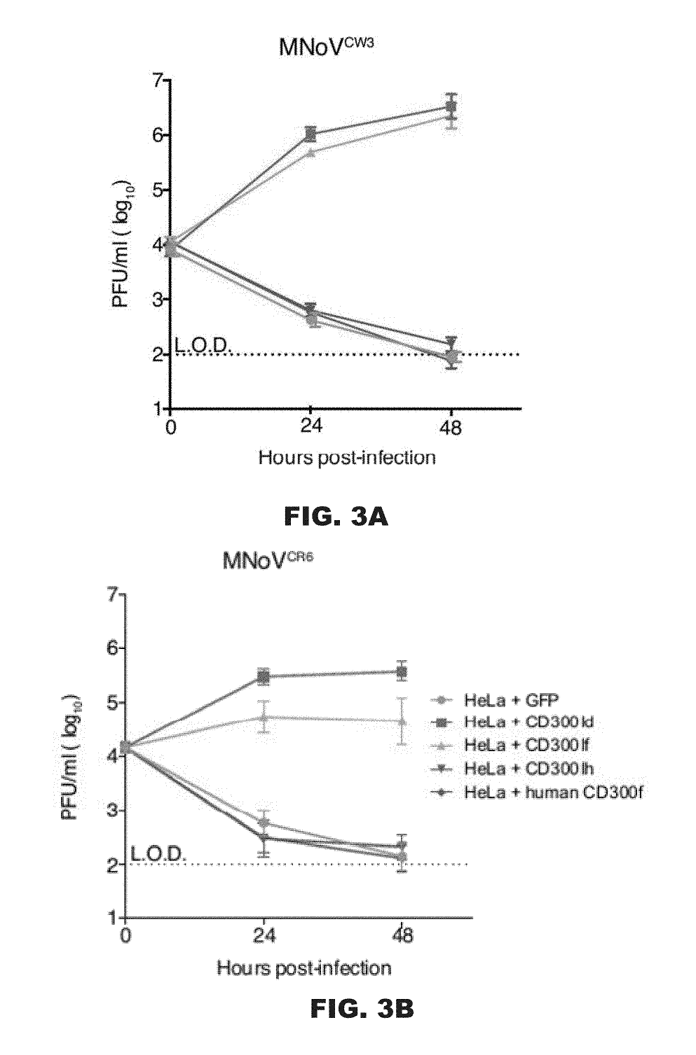

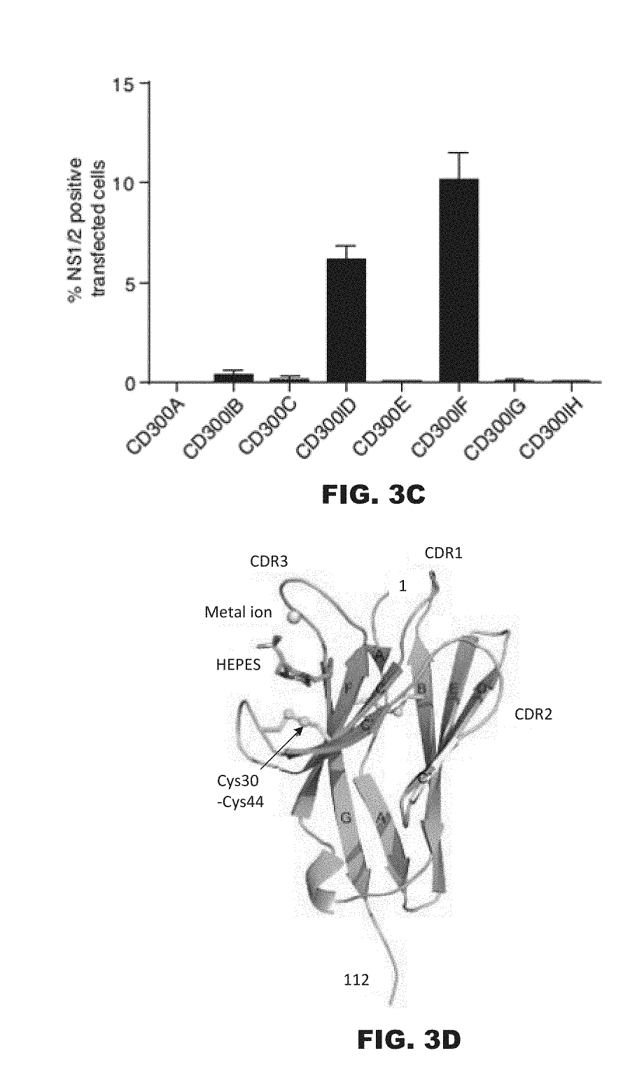

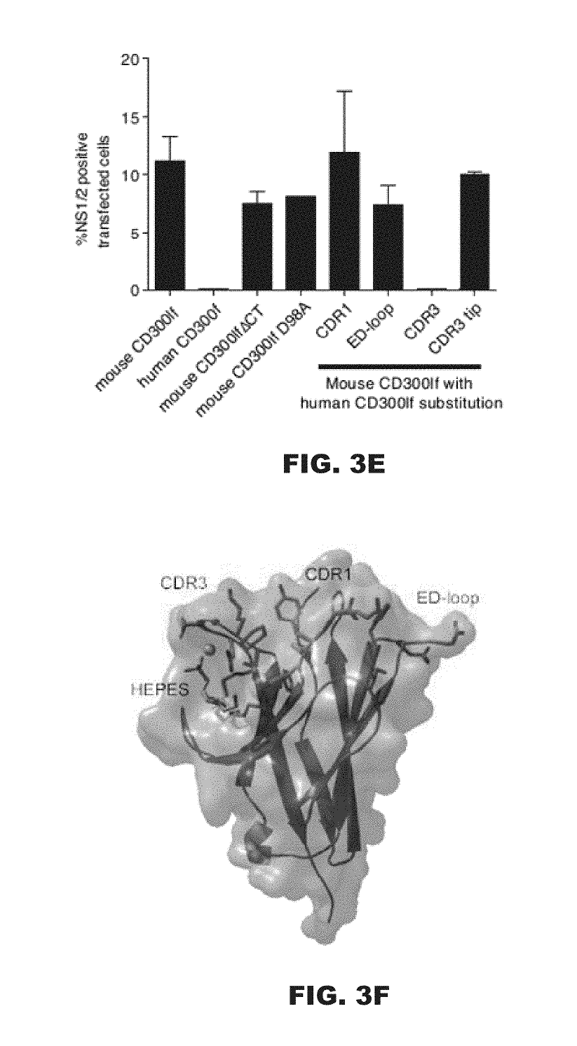

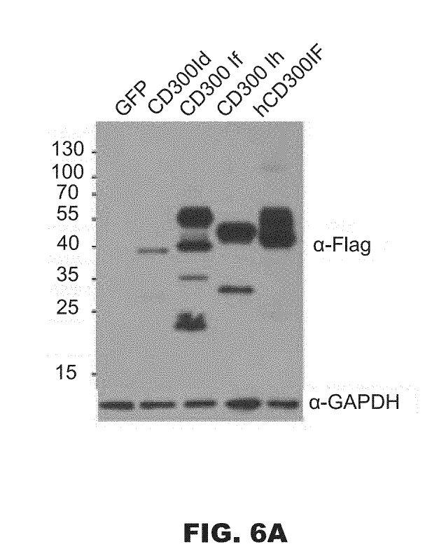

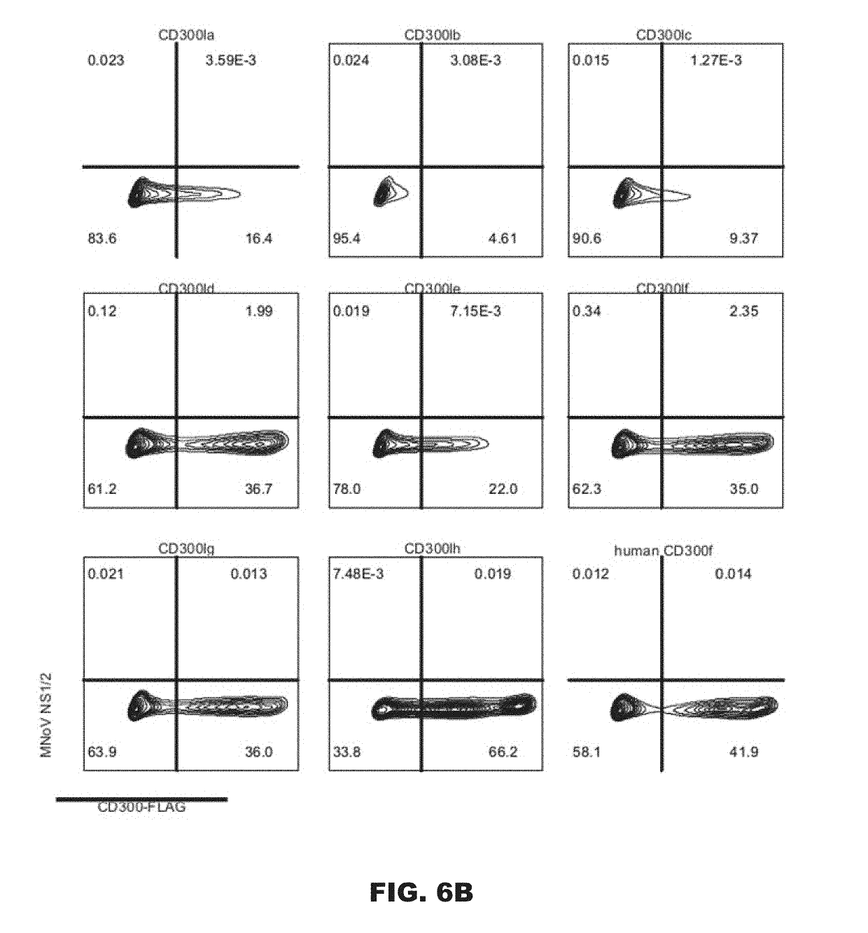

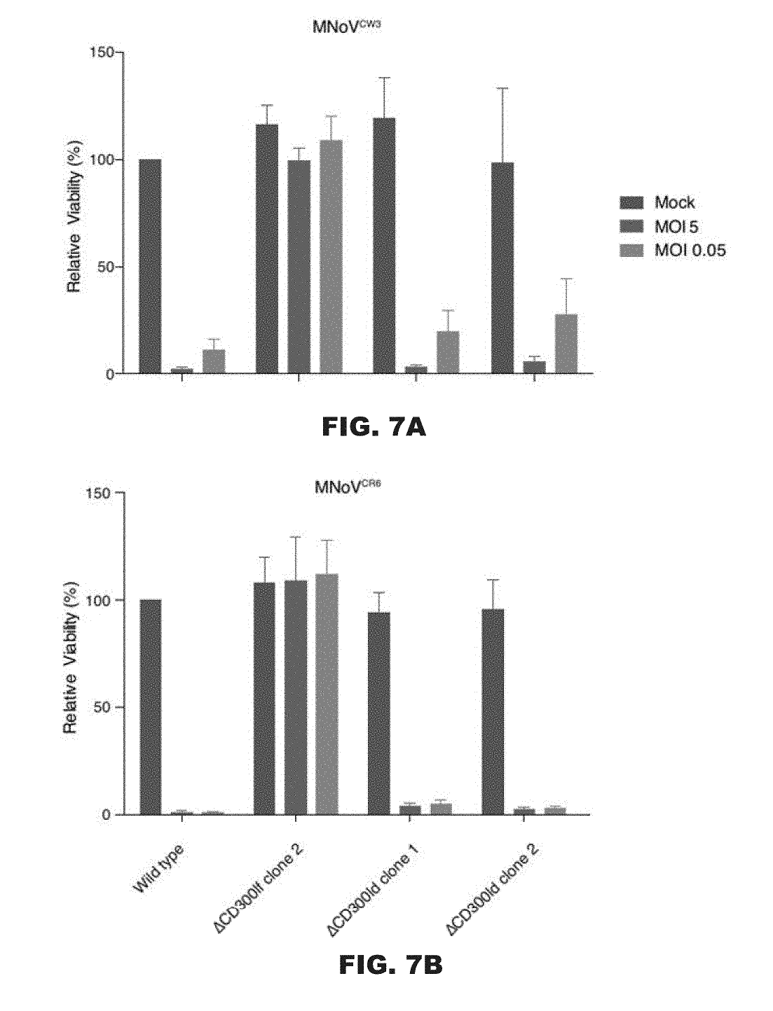

MNoV replicates in murine dendritic cells, macrophages, and B cells, but not in epithelial cells or human cells due to a restriction at viral entry [18, 19]. Therefore, we tested if expression of murine CD300lf was sufficient to confer susceptibility of HeLa cells to MNoV. As expected, HeLa cells transfected with a control GFP-expressing plasmid were unable to support MNoV^{CW3} or MNoV^{CR6} replication (FIG. 3A, FIG. 3B). In contrast, HeLa cells expressing murine but not human CD300lf were susceptible to both MNoV^{CW3} and MNoV^{CR6} (FIG. 3A, FIG. 3B). These data indicate that CD300lf expression is sufficient for MNoV growth in human cells and suggests that species-specific variation of CD300lf contributes to MNoV species restriction. In mice there are eight CD300 family members in addition to three copies of genes closely related to CD300lh. Therefore, we sought to determine whether other CD300 molecules are capable of functioning as an MNoV receptor. We transiently transfected cDNA expressing all eight CD300 family members into HeLa cells and assessed infectivity by the production of a viral non-structural protein by FACS. Only CD300lf and CD300ld conferred susceptibility to MNoV in HeLa cells (FIG. 3C, FIG. 6). We further confirmed that expression of CD300lf and CD300ld, but not CD300lh was sufficient for the production of infectious virions in human cells (FIG. 3A). However, in BV2 cells CD300ld is not required for MNoV infection, suggesting that MNoV utilization of CD300ld is not a requirement for infection but leaving open the potential for CD300ld to play a role in viral tropism in some circumstances in vivo (FIG. 7).

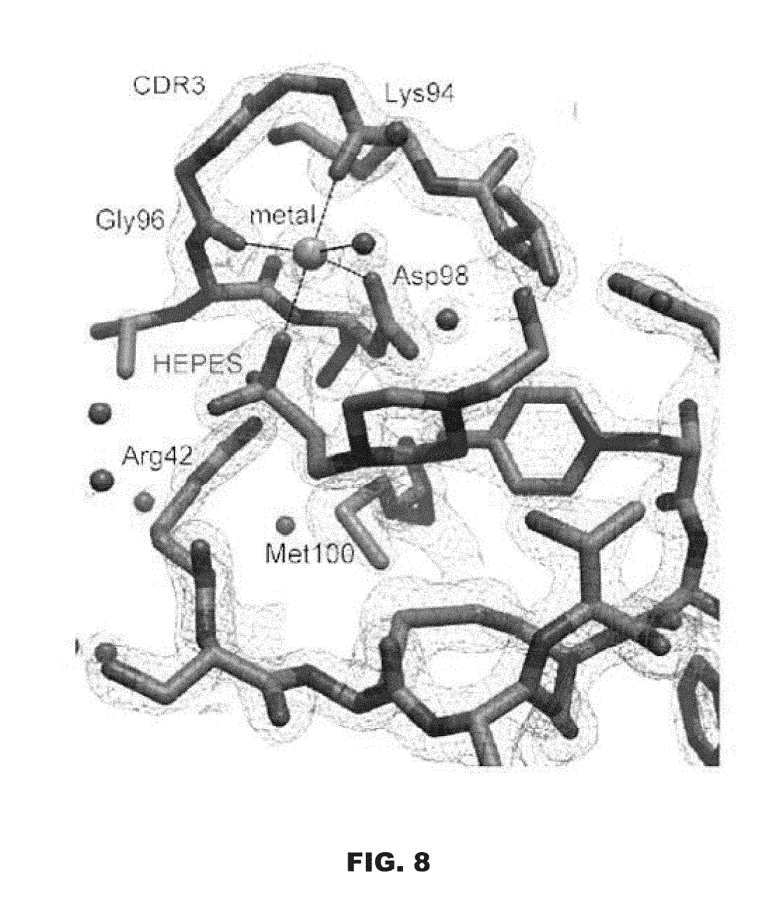

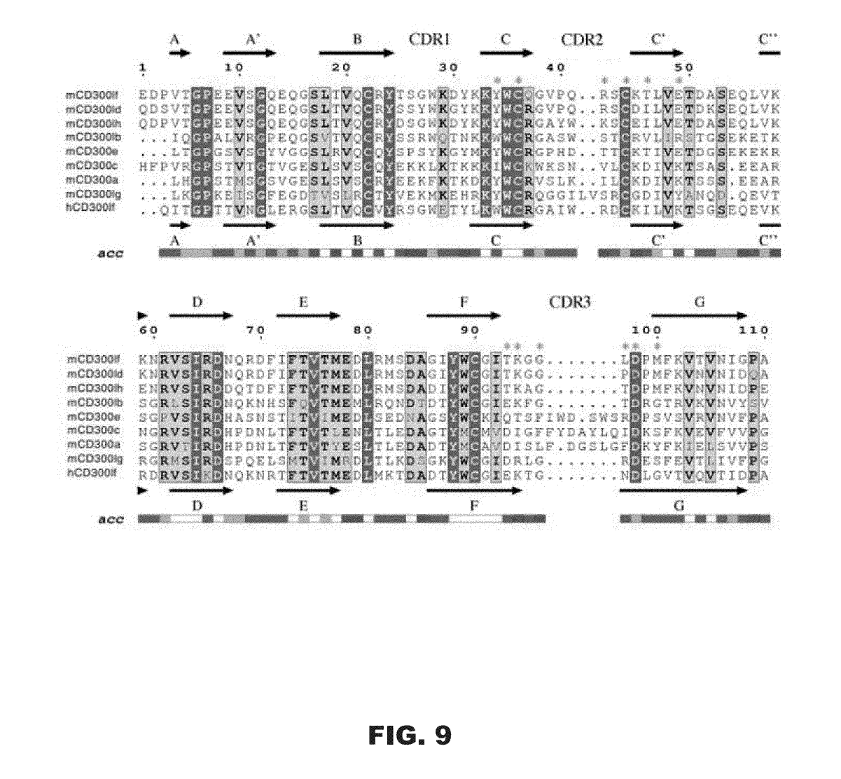

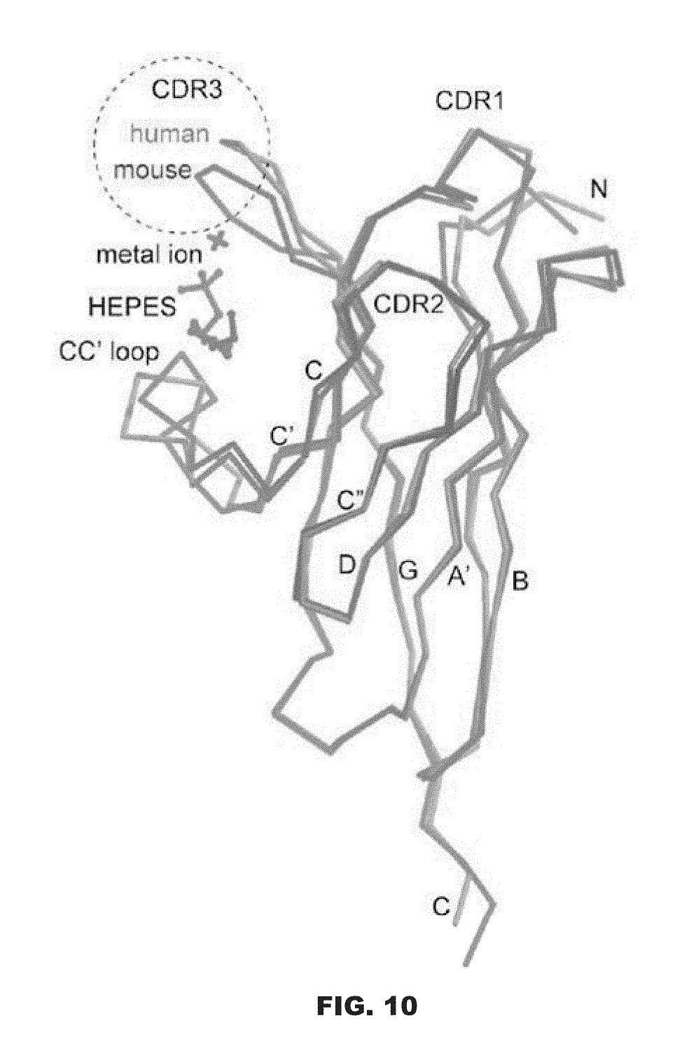

We next wanted to define the mechanism for MNoV entry via CD300lf and determine how MNoV discriminates between mouse and human proteins. Importantly, the intracellular domain of CD300lf was not required to make HeLa cells susceptible, indicating that species tropism is determined by the ectodomain (CD300lfΔCT, FIG. 3E). To gain insight into how the CD300lf lg-domain mediates viral entry, we determined the crystal structure at 1.6 Å resolution (FIG. 3D). Interestingly, density corresponding to a bound HEPES molecule and coordinated metal were clearly visible in a surface cleft formed between the CDR3 loop and the β-hairpin turn that connects the C—C′ β-strands. CD300lf has been reported to mediate the phagocytosis of apoptotic cells through the calcium-dependent binding of lipids such as phosphatidylserine (PS), phosphatidylglycerol (PG), and ceramide [20-22]. HEPES has some chemical resemblance to a phospholipid headgroup and serve as a surrogate ligand with the sulfate mimicking phosphate (FIG. 8). Our structure also reveals a metal coordinated primarily by CD300lf Asp98 and two CDR3 loop carbonyl oxygens. While mutation of murine CD300lf Asp98 has been shown to disrupt apoptotic cell surface binding, this mutant still allows MNoV^{CW3} infection of HeLa cells, suggesting that viral entry can occur in the absence of bound metal (FIG. 3E) [21, 23]. The Ig-domains of murine and human CD300lf share 59% sequence identity (FIG. 9), and structurally the largest variation occurs in CDR3 and the CC′-loop that form the apparent ligand and metal binding cleft not observed in the human structure (FIG. 10) [23]. Interestingly, substitution of human CDR3 into murine CD300lf blocks MNoV^{CW3} infection of HeLa cells while replacement of the solvent exposed CDR1 or ED-loop had no effect (FIG. 3E, FIG. 3F). These data provide a framework for understanding how MNoV discriminates between human and mouse CD300lf molecules.

Our work establishes that CD300lf is a functional MNoV receptor that mediates binding to the cell surface and is both necessary and sufficient for viral entry. Because MNoV serves as a model system for understanding how viruses persist and shape the immune system, the modulation of receptor availability either genetically or chemically may foster understanding of cell intrinsic and extrinsic mechanisms of immunomodulation, persistence, and tropism of MNoV across species. This work also enables the future study of MNoV replication in human cells, which may uncover novel mechanisms of viral replication and pathogenesis and allow a direct identification and mechanistic dissection of the cellular factors required for NoV replication across species. Additionally, our work has implications for understanding HNoV infections. HNoV binds to histo-blood group antigens (HBGA) and susceptibility to HNoV is correlated with host HBGA status [24, 25]. These reports are the foundation for the hypothesis that glycans function as HNoV receptors [10]. However, HBGA alone cannot, to date, explain species tropism or the entry barrier for HNoV. In contrast, our data indicate that murine CD300lf is sufficient to explain tropism for MNoV and more broadly suggest the possibility that other NoVs utilize proteinacious receptors in addition to carbohydrate-related attachment factors. It is possible that generation of small molecules that interrupt the interaction between NoV and such receptors may be therapeutic for acute and persistent NoV infection.

### Methods for the Example

Cell Culture:

BV2 cells and Hela cells (ATCC) were cultured in Dulbecco's Modified Eagle Medium (DMEM) with 10% fetal bovine serum (FBS), 1% HEPES, and 1% Penicillin/Streptomycin unless otherwise indicated. For BV2 cells, 2.5 μg/ml of puromycin (Sigma Aldrich), 4 μg/ml blasticidin (Invitrogen), and 300 μg/ml hygromycin (Invitrogen) were added as appropriate. BV2 cells were karyotyped (Clinical Genomics Research Unit, Washington University) by the GTW-banding method and determined to be hypertriploid (karyotype: 59˜66,XX,-X/Y,-2,i(2)(A1), +3,-4,-6,add(8)(A1)x2,-9,-10,add(14)(A1),+15,+16,-17,-19,+6-10mar[cp10])

CRISPR Screen:

BV2 cells were transduced with pXPR_101 (Addgene plasmid 52962) and selected with blasticidin for eleven days [1]. Cas9 activity was assessed by transducing parental BV2 or BV2-Cas9 cells with pXPR_011 (Addgene plasmid 59702), expresses eGFP and a sgRNA targeting eGFP at an MOI<1 [1]. Cells were transduced for 2 days and subsequently, selected for five days with puromycin and the frequency of eGFP expression was assessed by flow cytometry. The mouse ASIAGO sgRNA CRISPR library contains six independent genome wide pools which each pool containing a unique sgRNA targeting each of 20,077 mouse genes. Four ASIAGO pools were independently used to generate the BV2 library (Doench et al submitted). Each ASIAGO pool was delivered by lentiviral transduction of 5×10⁷ BV2 cells at an MOI ˜0.2. This equates to 1×10⁷ transduced cells, which is sufficient for the integration of each sgRNA 500 independent times. Two days post transduction, puromycin was added to the media and transduced cells were selected for five days. For each experimental condition, 1×10⁷ BV2-Cas9 cells containing sgRNAs were seeded in a T175 tissue culture flask. Cells were infected with either MNoV^{CW3} or MNoV^{CR6} at MOls of 0.05 and 5. Mock infected cells were harvested 48 hrs after seeding and served as a reference for sgRNA enrichment analysis. Ten days post-infection genomic DNA was isolated from surviving cells using a Qiamp DNA mini kit according to manufacturer instructions (Qiagen).

CRISPR Screen Sequencing and Analysis:

Illumina sequencing and STARS analysis was performed as described previously (Doench et al, in press). Briefly, genomic DNA was aliquoted into multiple wells of a 96-well plate with up to 10 μg of DNA in 50 μl total volume. A PCR mastermix consisting of ExTaq DNA polymerase (Clontech), ExTaq buffer, dNTP, P5 stagger primer, and water was generated. 40 μl of PCR master mix, and 10 μl of a barcoded primer was added to each well containing 50 μl of DNA. Samples were PCR amplified as follows: 95° C. for 1 min., followed by 28 cycles of 94° C. for 30 sec., 52.5° C. for 30 sec., 72° C. for 30 sec. with a final 10 min. at 72° C. PCR product was purified with Agencourt AMPure XP SPRI beads according to the manufacture's protocol (Beckman Coluter). Samples were sequenced on an Illumina HiSeq 2000. Barcodes in the P7 primer were deconvuluted and the sgRNA sequence was mapped to a reference file of sgRNAs in the ASIAGO library. To normalize for different numbers of reads per condition, read counts per sgRNA were normalized to 10⁷ total reads per sample. This normalized value was then log-2 transformed. sgRNAs that were not sequenced were arbitrarily assigned a read count of 1. sgRNA frequencies were then analyzed using STARS software, which is available at broadinstitute.org/rnai/public/software/index (Doench et al, in press). STARS computes a score for each gene of rank-ordered sgRNA hits that was above XX % of total sequenced sgRNAs. A STAR score was only assigned to genes that scored above this threshold in at least two of four independent genome wide pools.

Generation of MNoV Stocks:

MNoV^{CW3} (Gen bank accession no. EF014462.1) and MNoV^{CR6} (Gen bank accession no. JQ237823) were generated by transfecting MNoV cDNA clones into 293T cells as described previously [2]. Briefly, 293T cells were frozen 48 hr after transfection, and the supernatant was clarified to generate the P0 stock. To generate a P1 stock, BV2 cells were infected at an MOI of 0.05 for 48 hrs. This P1 stock was then used to infect BV2 cells at an MOI of 0.05 for 24-36 hrs to generate a P2 stock, which was used for all subsequent experiments. To isolate P2 MNoV, P1-infected BV2 cells were frozen, thawed, the cell lysate and supernatant was centrifuged at 3,000 rpm for 20 min., and the supernatant was filtered through a 0.22 μm filter, and then pelleted by centrifugation through a 30% sucrose cushion at 154,000 g (Rmax) for 3 hr. Pelleted MNoV was resuspended in media, aliquoted, and stored at −80° C. P2 stocks were titered three independent times prior to use.

Generation of CD300lf Knockout and Complemented Cells:

CD300lf and CD300ld BV2 knockout cells were generated at the Genome Engineering and iPSC center at Washington University School of Medicine. BV2 cells were nucleofected with Cas9 and a CD300lf-specific sgRNA SEQ ID NO:1 (5′ GTGCAGTGCCGATATACCTC 3′) or CD300ld-specific sgRNA SEQ ID NO:2 (5′ AGATATTCCTCATACTGGAA 3′). Cells were then single cell sorted and genomic DNA was extracted. DNA was amplified using the following forward and reverse primers: SEQ ID NO:3 (5′ AGGATGTCGAGGGATGGCAGGCAGC 3′) and SEQ ID NO:4 (5′ TGCCAGCATCGCTCATCCTCAGATCC 3′), respectively, for CD300lf and SEQ ID NO:5 (5′ CACCGAGATATTCCTCATACTGGAA 3′) and SEQ ID NO:6 (5′ AAACTTCCAGTATGAGGAATATCTC 3′) for CD300ld. Clones were screened for frameshifts by sequencing the target region with Illumina MiSeq at approximately 500× coverage. Two ΔCD300lf and two ΔCD300ld BV2 clones were selected for subsequent experiments (clone 1 and clone 2).

The targeted CD300lf mutations in these two clones are shown below (SEQ ID NOs: 7-131:

| | |
|---|---|
| Q E Q G S L T V Q C R Y T S G W K D Y K | |
| CAGGAGCAGGGCTCCTTGACAGTGCAGTGCCGATATACCTCAGGCTGGAAGGATTACAAG | (WT) |
| CAGGAGCAGGGCTCCTTGACAGTGCAGTGCCGATAT--CTCAGGCTGGAAGGATTACAAG | (Clone 1.1) |
| CAGGAGCAGGGCTCCTTGACAGTGCAGTGCCGATATA-CTCAGGCTGGAAGGATTACAAG | (Clone 1.2) |
| CAGGAGCAGGG-----------------------------------------------AG | (Clone 1.3) |
| CAGGAGCAGGGCTCCTTGACAGTGCAGTGCC-------CTCAGGCTGGAAGGATTACAAG | (Clone 2.1) |
| CAGGAGCAGGGCTCCTTGACAGTGCAGTGCCGATATA-CTCAGGCTGGAAGGATTACAAG | (Clone 2.2) |

ΔCD300lf BV2 clones were complemented by lentiviral transduction of pCDH-CMV-MCS-EF1-Hygro (System Biosciences, Inc) containing codon-optimized CD300lf with a C-terminal FLAG. Two days post transduction cells were selected with hygromycin for five days.

The targeted CD300ld mutations in these two clones are shown below (SEQ ID NOs: 14-19):

| | |
|---|---|
| E Q G S L T V Q C R Y S S Y W K G Y K K | |
| GAGCAGGGCTCCTTGACAGTGCAGTGCAGATATTCCTCATACTGGAAGGGTTACAAGAAG | (WT) |
| GAGCAGGGCTCCTTGACAGTGCAGTGCAGATATTCCTCA--------GGGTTACAAGAAG | (clone 1.1) |
| GAGCAGGGCTCCTTGACAGTGCAGTGCAGATATTCCTCATACTG-AAGGGTTACAAGAAG | (clone 1.2) |
| GAGCAGGGCTCCTTGACAGTGCAGTGCAGATATTCCTCATACTG-AAGGGTTACAAGAAG | (clone 2.1) |
| GAGCAGGGCTCCTTGACAGTGCAGTGCAGATATTCCTCATAC-GGAAGGGTTACAAGAAG | (clone 2.2) |

Western Blots:

Cells were lysed in RIPA buffer (50 mM tris-HCl ph 7.4, 1% Igepal, 0.5% sodium deoxycholate, 0.1% sodium dodecyl sulfate, 150 mM NaCl, 2 mM EDTA) supplemented with HALT protease and phosphatase inhibitor cocktail for 30 minutes on ice. Lysates were clarified by centrifugation, subjected to SDS-PAGE, and transferred to PVDF membrane. Membranes were probed with α-FLAG-M2-HRP (Sigma; 1:10,000) and α-GAPDH-HRP (Sigma, 1:75,000).

MNoV Growth Curves:

For MNoV growth curves, 5×10⁴ BV2 or Hela cells were infected in suspension with MNoV^{CW3} or MNoV^{CR6} at an MOI of 0.05 in a well of a 96-well plate. For BV2 cell growth curves, plates were frozen 0, 12, and 24hpi. For Hela cell growth curves, plates were frozen at 0, 24, and 48 hpi. Total cell lysate was used in subsequent plaque assays. All infections were done in triplicate in each of at least three independent experiments.

MNoV Plaque Assays:

BV2 cells were seeded at 1×10⁶ cells/well of a six-well plate. 16-24 hours later, media was removed and 10-fold dilutions of cell lysate is applied to each well for 1 hour with gentle rocking. Viral inoculum was removed and 2 ml of overlay media was added (MEM, 10% FBS, 2 mM L-Gluatmine, 10 mM HEPES, and 1% methylcellulose). Plates were incubated for 2-3 days prior to visualization with crystal violet solution (0.2% crystal violet and 20% ethanol).

Viral RNA Transfection:

MNoV^{CW3} RNA was extracted from cell-free viral preparations using TRIzol (Invitrogen) according to manufacturer instructions. Purified viral RNA was plagued to ensure complete inactivation of MNoV. 10 μg of vRNA was transfected using Lipofectamine 2000 (Invitrogen) according to the manufacturer's protocol. Transfected cells were frozen 12 hours later. Each condition was assayed by plaque assay in triplicate in three independent experiments.

Generation of sCD300lf: A cDNA fragment encoding the extracellular domain of mouse CD300lf (residues 20-128 of GenBank Accession No. AAH57864.1) was inserted into the E. coli T7 expression vector pET-28 downstream of a methionine codon. BL21-DE3-RIL cells (Agilent) carrying the plasmid were grown at 37° C. in LB to an OD600 of 0.8 before 4 hours of induction with 1 mM IPTG. The cells were harvested and stirred on ice for 45 minutes in 50 mM Tris-HCL pH 7.0 containing 250 μg/ml lysozyme, 100 μg/ml DNasel, and 50 mM MgCl2. After sonication the lysate was spun 20 minutes at 10,000 g and the supernatant discarded. The recovered inclusion body pellet was washed several times with 50 mM Tris-HCL pH 7.0, 150 mM NaCl, and 10 mM DTT containing 1% Triton-X100. A final wash was performed in the same buffer without detergent. For a single refolding, approximately 50 mg of inclusion body protein was denatured in 3 ml of 6 M guanidine hydrochloride buffered with 20 mM Tris-HCL pH 8.0 and reduced by addition of 20 mM 2-mercaptoethanol for 1 hour. The denatured protein was further diluted to 10 ml using 3 M guanidine hydrochloride containing 20 mM Tris-HCL pH 8.0. The protein was refolded by rapid dilution into 1 liter of 4° C. buffer consisting of 0.4 M NDSB-201 (Santa Cruz Biotechnology), 100 mM Tris-HCl, 10 mM EDTA, 0.2 mM PMSF, 5 mM reduced glutathione, and 0.5 mM oxidized glutathione at a final pH of 8.3. After 24 hours the natively-folded protein was collected above a 10 kD membrane (Millipore number PLGC07610) in a stirred cell concentrator and separated from aggregate by passage over Superdex 200. Fractions containing the correct molecular weight were pooled and stored at 4° C. in sizing buffer consisting of 25 mM HEPES pH 7.5, 150 mM NaCl with 0.01% sodium azide.

MNoV Neutralization with α-CD300lf and sCD300lf:

MNoV neutralization was assessed by measuring cytotoxicity. 2.5×10⁴ BV2 cells were plated per well of a 96-well plate. For antibody-mediated neutralization, cells were then incubated for 1 hour at 37° C. with polyclonal α-CD300lf (R&D systems AF2788) or an isotype control (R&D systems AB-108-C) at a final concentration from 0.01 to 10 μg/ml. MNoV^{CW3} was then added at an MOI of 5 in a total volume of 100 μl. After 24 hours at 37° C., 25 μl of CellTiter-Glo (Promega) was added per well to measure cellular ATP concentrations. For sCD300lf neutralization, the assay was performed similarly except that 1.25×10⁵ PFU of MNoV^{CW3} was incubated with sCD300lf or a control protein (Domain III of West Nile Virus Envelope) at a final concentration from 0.01 μg to 10 μg/ml prior to addition of BV2 cells [3]. Relative luminescence units were detected on Molecular Devices SpectraMax M2 plate reader. All conditions were normalized to an uninfected control. Each condition was done in quadruplicate in each of three independent experiments.

Secondary Validation of Screening Hits:

sgRNA were cloned into plentiCRISPRv2 (addgene plasmid #52961). BV2-Cas9 cells were individually transduced with lentiviruses expressing four unique sgRNA per gene and selected with puromycin for one week post transduction. Cells were counted, infected with MNV^{CW3} or MNV^{CR6} at an MOI 0.05, and incubated for two days before assessing cellular viability as described above.

Quantitative PCR:

MNoV^{CW3} quantitiatve PCR (qPCR) was performed as previously described [4]. Briefly, 5 μl of extracted viral RNA was used for cDNA synthesis using random hexamers and ImProm Reverse Transcriptase (Promega, Madison, Wis.) in 20 μl total volume. TaqMan qPCR was performed in triplicate on each sample and standard with forward primer SEQ ID NO:20 (5′ CACGCCACCGATCTGTTCTG 3′), reverse primer SEQ ID NO:21 (5′ GCGCTGCGCCATCACTC 3′), and probe.

MNoV Binding Assay:

MNoV^{CW3} binding to BV2 cells was performed for 1 hour at 4° C. in 0.5 ml complete growth media. sCD300lf was incubated with MNoV^{CW3} for 30 minutes at room temperature prior to addition to cells. α-CD300lf was incubated with cells for 30 minutes at 4° C. prior to the addition of MNoV^{CW3}. BV2 cells were used at final concentration of 2×10⁶ cells/ml, and MNoV^{CW3} was used at a final concentration of 1.6×10⁹ copies/ml (2.5×10⁶ PFU/ml). Cells were centrifuged at 500 g for 5 min at 4° C. to remove unbound virus. Cells were washed with four 1.0 ml washes with PBS containing 2.5% FBS and 2 mM EDTA. The cell pellet was resuspended in 100 μl PBS, and RNA was extracted with the ZR Viral RNA kit (Zymo Research) according to manufacturer instructions. qPCR was performed as described above. Binding experiments were done in triplicate in each of at least three independent experiments. A condition containing virus but no cells was used to determine assay background.

Kifunensine Treatment:

To determine the effect of glycans on MNoV binding, parental BV2 cells, ΔCD300lf BV2 clone 1 cells complemented with an empty lentivirus, and ΔCD300lf BV2 clone 1 cells complemented with CD300lf were cultured in the presence of 1 μg/ml of kifunensine (Santa Cruz Biotechnology) for at least 48 hours prior to use. Kifunensine activity was determined by staining cells with FITC-conjugated wheat germ agglutinin (WGA) at 1 μg/ml final concentration (Sigma Aldrich). The reduction in glycosylation was determined by comparing the mean fluorescence intensity (MFI) with and without kifunensine treatment. WGA binding was determined at the time of each binding assay.

Bmdm Infection:

BL6 myeloid progenitors were obtained from mice (FIG. 2E) or from HoxB8-immortalized progenitors (FIG. 2D) as previously described [5].HoxB8-immortalized progenitors were maintained in RPMI 1640 supplemented with 1 μM β-estradiol and 25 ng/ml GM-CSF. Progenitors were differentiated into macrophages by washing the cells in PBS and plating 1×10⁶ cells into BMDM media (Dulbeco's Modified Eagle Medium, 10% fetal bovine serum, 5% horse serum, 10% CMG14-12 [6], 1 mM sodium pyruvate, 2 mM L-glutamine). After 7 days of differentiation, macrophages were dissociated and 5×10⁶ cells/well were seeded into a 6-well dish. Three days later, MNoV^{CW3} was added to cells at an MOI of 5.0. One day post infection, MNoV NS1/2 expression was detected by flow cytometry.

FACS Assay for MNoV Infection:

Infected cells were fixed and permeabilized with Cytofix/Cytoperm (BD biosciences) Rabbit polyclonal antibody targeting the MNoV nonstructural protein NS1/2 antibody was added at a 1:5000 dilution (Gift from Vernon Ward). Cells were incubated for 30 min. at room temperature. Cells were then washed and DyLight 649 donkey anti-rabbit IgG was added and incubated for 30 min. Cells were then washed and analyzed on an LSRII flow cytometer (BD). At least 20,000 events were collected per condition. Each experiment was performed in triplicate in each of three independent experiments.

Mouse Infections:

B6.129S(Cg)-Stat1^{tm1Div}/J (STAT1^{-/-}) [7] mice and C57BL/6J (Jackson Laboratories, Bar Harbor, Me.) and Fut2^{-/-} (B6.129X1-Fut2^{tm1Sdo}/J) mice (a generous gift from Dr. Alexander V. Chervonsky) were housed in a specific-pathogen free environment at Washington University. The care and use of all animals was approved by and in accordance with the Washington University Animal Studies Committee. For the challenge of STAT^{-/-} mice, MNoV^{CW3} was incubated with sCD300lf or WNV E DIII at a final concentration of 10 μg/ml for 30 minutes at room temperature. STAT1^{-/-} mice were challenged with 25 μl PO of 10³ or 10⁵ PFU of MNoV^{CW3} diluted in D10. STAT1^{-/-} mice were singly housed immediately after inoculation and monitored daily for survival for 28 days. Experiments were performed three independent times with gender-balanced littermate controls at both 10³ and 10⁵ PFU challenge doses.

To isolate in vivo derived MNoV^{CW3}, five 7-8 week old C57BL/6J were inoculated PO with 10⁶ PFU MNoV^{CW3} in 25 μl PBS. Three days post inoculation, mice were sacrificed and spleens were harvested. Spleens were processed by bead beating with 1 mm silica beads (Biospec, Bartlesville, Okla.) for 1 minute. The spleen extract was centrifuged for 5 minutes at 3,000 g, the supernatant was filtered through a 0.22 μm filter, frozen at −80° C., and titered on BV2 cells as described previously.

Fut2^{-/-} and Fut2^{+/+} sex-matched littermates were inoculated with 10⁶ PFU of MNoV^{CW3} at 8-10 weeks of age PO. At day 7 post-infection, tissues were harvested and flash frozen in a bath of ethanol and dry ice and stored at −80° C. prior to processing. RNA from tissues was isolated with Tri Reagent (Invitrogen) according to the manufacturer's protocol. MNoV^{CW3} was detected via qPCR as described above.

Spleen-Derived MNoV Neutralization:

75 PFU of either BV2 cell-derived or spleen-derived MNoV^{CW3} was incubated for 30 min. at room temperature with either sCD300lf or WNV E DIII at 10 μg/ml final concentration in 100 μl total volume of D10. Each sample was then added to a confluent monolayer of BV2 cells in a 6-well plate and incubated for 1 hr. at room temperature. Unbound virus was then washed off with three 2-ml PBS washes. Complete media containing methylcellulose was then added and plaque formation was assessed as described previously. Each experiment was performed in triplicate in each of three independent experiments.

Complementation of HeLa Cells:

1 μg of pCDNA3.1 (CD300la and GFP), pCDNA3.4 (CD300ld, CD300lf, CD300lh, and human CD300f), or pCMV6 (CD300lb, CD3001c, CD3001e, and CD300lg) plasmids were transiently transfected into HeLa cells with Trans-It LT1 (Mirus Bio) according to manufacturer instructions. One day post-transfection, cells were trypsinized, infected for viral growth curves (described above), or for FACS analysis. At least three independent experiments were performed per condition.

Crystallization and Structure Determination:

Purified sCD300lf protein at 40 mg/ml in 20 mM HEPES pH 7.4 and 20 mM NaCl was mixed with an equal volume of reservoir solution containing 35% (v/v) 2-methyl-2,4-pentanediol, 50 mM sodium chloride, 120 mM Tris-HCL at pH 7.25 in hanging drops. Crystal were prepared for analysis by flash cooling to 100° K in drop solution. Diffraction data were collected on a RUH3R rotating anode X-ray generator fitted with Varimax Optics and an Raxis-IV image plate area detector (Rigaku). The resulting crystals contained a single monomer per asymmetric unit and belonged to the orthorhombic space group P212121 with unit cell dimensions a=27.63 Å, b=53.35 Å, and c=70.52 Å. The diffraction data were integrated and scaled with HKL2000 [9]. The structure was solved by the molecular replacement method using Phaser [10] and coordinates of the N-terminal domain from the polymeric-immunoglobulin receptor (PDB ID:1XED) as the search model. Atomic refinement was undertaken using Phenix with model building using COOT [11, 12]. The Ramachandran plot for the final sCD300lf structure showed 100% of the residues in the most favorable regions with no residues in the additionally allowed or outlier regions. Summaries of the data collection and refinement statistics are provided in Table 6. All structure figures were created using PyMOL (pymol.org). The atomic coordinates were deposited in the Protein Data Bank under accession code 5FFL.

Binding of HEPES and a Metal Ion to sCD300lf:

The mouse sCD300lf structure adopts a typical V-type immunoglobulin fold. Refinement of the atomic model revealed a bound HEPES molecule, presumably captured from the size exclusion buffer during protein purification. We speculate that HEPES may be serving as a place-holder for a physiological ligand. First. HEPES resembles a phospholipid headgroup with the sulfate group mimicking a lipid phosphate group. Superposition of PS onto HEPES in the structure suggests the orientation within the binding site would allow the fatty acid chain of the lipid to contact several hydrophobic residues in the cleft and extend away from the CD300lf bearing cell. CD300lf is reported to bind the physiological ligands in a calcium-dependent manner. The HEPES in our structure is held in the binding site by a metal ion. Three oxygen atoms from the CDR3 loop coordinate the metal binding: carbonyl oxygens from residues Lys94 and Gly96, and one from the side chain of Asp98. The side chain of Asp98 has been reported to be required for CD300lf phosphatidylserine recognition [13].

The coordination geometry (octahedral) and distance between atoms in our structure is consistent with either a sodium or calcium ion [14]. We modeled the density as a sodium because it was present in the buffers used for purification, refolding, and crystallization, while calcium was not. Further, the b-factor of the refined sodium atom (30.6 Å²) matches closely with that of its environment (31.3 Å²).

Site Directed Mutagenesis:

We used the Q5 site-directed mutagenesis kit as directed to generate mutations in pCDNA3.4 CD300lf. All constructs were sequenced confirmed. CD300lf^{CT} (E230STOP) and CD300lf^{CDreq3} (TKGGLDPMFK to EKTGNDLGVT) (SEQ ID NO: 26-27) are shown in FIG. 3.

Statistical Analysis:

Error bars represent the standard error of the mean (SEM) unless otherwise stated. All pairwise comparisons analyzed statistically are indicated by a horizontal line with a symbol representing the p-value. The absence of a line between two groups indicates a statistical comparison was not performed. For pairwise comparisons, Mann-Whitney tests were performed for non-normally distributed data, and student T-tests were performed for normally distributed data. A p-value <0.05 was considered significant. All statistical analysis was performed with GraphPad Prism 6 unless otherwise indicated.

TABLE 1
MNoV^{CW3} MOI 5 STARS output.

| | | | Most | | | | | |
|---|---|---|---|---|---|---|---|---|
| Gene | No of | | enriched | STARS | Average | | | |
| Symbol | spacers | Ranks of spacers | spacer | Score | Score | p-value | FDR | q-value |
| Cd300lf | 4 | 1; 4; 6; 7 | 4 | 16.22415466 | 10.02755566 | 0 | 0 | 0 |
| G3bp1 | 4 | 20; 31; 65; 10334 | 3 | 8.662964652 | 5.901025177 | 0 | 0 | 0 |
| Kmt2d | 4 | 5; 71; 72; 730 | 3 | 8.529745438 | 6.40089237 | 0 | 0 | 0 |
| Kdm6a | 4 | 33; 60; 99; 22147 | 3 | 8.114984736 | 5.454853922 | 0 | 0 | 0 |
| Cd300lh | 4 | 2; 3; 30226; 58302 | 2 | 8.069912369 | 6.033995903 | 0 | 0 | 0 |
| Myo10 | 4 | 41; 45; 6715; 51798 | 2 | 5.718188054 | 4.20257591 | 0.006146654 | 0.038928808 | 0.038928808 |
| Rps8 | 4 | 410; 580; 669; 1917 | 3 | 5.628735337 | 3.608455956 | 0.007531815 | 0.040886998 | 0.040886998 |
| Gapdh | 4 | 239; 628; 722; 2999 | 3 | 5.529693661 | 3.629789146 | 0.008916977 | 0.04235564 | 0.041304745 |
| Rpl29 | 4 | 417; 475; 734; 5151 | 3 | 5.508283115 | 3.623331511 | 0.009782703 | 0.041304745 | 0.041304745 |
| Pik3ap1 | 4 | 216; 280; 5420; 34740 | 2 | 4.132865242 | 3.050513324 | 0.125876547 | 0.47833088 | 0.47833088 |
| Smarce1 | 4 | 70; 300; 7248; 20583 | 2 | 4.073157718 | 3.264141029 | 0.145615098 | 0.503033976 | 0.503033976 |
| Rps4x | 4 | 165; 396; 1829; 2165 | 2 | 3.833061456 | 2.958678168 | 0.248896199 | 0.788171298 | 0.701952212 |
| Prkaa1 | 4 | 43; 398; 18692; 63456 | 2 | 3.828707606 | 3.247509755 | 0.251147087 | 0.734122254 | 0.701952212 |
| Tbck | 4 | 112; 421; 15706; 66676 | 2 | 3.7801617 | 3.01592697 | 0.280928058 | 0.762519015 | 0.701952212 |
| Med9 | 4 | 343; 428; 10117; 50252 | 2 | 3.765915107 | 2.767660556 | 0.28967189 | 0.733835454 | 0.701952212 |
| Sdhd | 4 | 382; 433; 11445:21351 | 2 | 3.75588168 | 2.739579689 | 0.295558826 | 0.701952212 | 0.701952212 |
| Rps20 | 4 | 383; 479; 3077; 7319 | 2 | 3.668691025 | 2.695424875 | 0.357977664 | 0.800185367 | 0.786069508 |
| Psmb4 | 4 | 471; 488; 1483; 2836 | 2 | 3.652621164 | 2.643202575 | 0.372348714 | 0.786069508 | 0.786069508 |
| Rplp2 | 4 | 284; 510; 2185; 3806 | 2 | 3.614561907 | 2.732487335 | 0.403774565 | 0.80754913 | 0.80754913 |
| Polr2e | 4 | 181; 536; 3357; 42584 | 2 | 3.571658119 | 2.808010344 | 0.446801143 | 0.848922171 | 0.812844487 |
| Rpl7a | 4 | 157; 546; 2568; 2840 | 2 | 3.555712222 | 2.830730126 | 0.465933685 | 0.843118097 | 0.812844487 |
| Cflar | 4 | 481; 565; 3650; 6190 | 2 | 3.526209287 | 2.57551684 | 0.497099818 | 0.858626959 | 0.812844487 |
| Fez1 | 4 | 123; 566; 45376; 47453 | 2 | 3.524684304 | 2.867934837 | 0.499004415 | 0.824442077 | 0.812844487 |
| Atp5g3 | 4 | 351; 573; 39016; 52088 | 2 | 3.514084817 | 2.636804637 | 0.513375465 | 0.812844487 | 0.812844487 |
| Polr2l | 4 | 149; 603; 1370; 1833 | 2 | 3.470089059 | 2.799209673 | 0.568175916 | 0.863627392 | 0.819759072 |
| Rpl34 | 4 | 199; 608; 1140; 1153 | 2 | 3.462971474 | 2.733227358 | 0.578651199 | 0.845720983 | 0.819759072 |
| Ubxn4 | 4 | 232; 610; 66701; 71089 | 2 | 3.460140943 | 2.698765302 | 0.582460393 | 0.819759072 | 0.819759072 |
| Olfr1254 | 4 | 62; 630; 23746; 64575 | 2 | 3.432339352 | 2.970019528 | 0.621244914 | 0.843118097 | 0.843118097 |
| Rps24 | 4 | 144; 647; 1165; 2524 | 2 | 3.409398795 | 2.776235455 | 0.653276773 | 0.85601784 | 0.85601784 |
| Birc3 | 4 | 32; 703; 13365; 58427 | 2 | 3.33791268 | 3.066181511 | 0.770669206 | 0.976180994 | 0.904683577 |
| Rpl37 | 4 | 525; 705; 1615; 1837 | 2 | 3.335467103 | 2.461500831 | 0.775344126 | 0.950421832 | 0.904683577 |
| Rrm2 | 4 | 184; 710; 3607; 25565 | 2 | 3.32938366 | 2.683328087 | 0.786858281 | 0.934394208 | 0.904683577 |
| BC061237 | 4 | 298; 717; 1258; 2947 | 2 | 3.320939081 | 2.575341896 | 0.801315903 | 0.922727404 | 0.904683577 |
| Cct2 | 4 | 496; 720; 5236; 5277 | 2 | 3.317345416 | 2.464540058 | 0.809453727 | 0.904683577 | 0.904683577 |
| Hspa5 | 4 | 678; 760; 3240; 3615 | 2 | 3.270823568 | 2.374904891 | 0.904164142 | 0.981663925 | 0.975139238 |
| Hnrnpa3 | 4 | 416; 768; 4458; 12288 | 2 | 3.261816408 | 2.47431158 | 0.92381612 | 0.975139238 | 0.975139238 |
| Eif5a | 4 | 765; 791; 2619; 57998 | 2 | 3.236439205 | 2.332214981 | 0.976711973 | 1 | 0.9883127 |
| Tnfrsf1a | 4 | 699; 796; 46519; 63199 | 2 | 3.231021117 | 2.348553197 | 0.9883127 | 0.9883127 | 0.9883127 |

p-value = 0 means <8.65725911177e−05,
FDR = 0 means <0.000657951692494

TABLE 2
MNoV^{CW3} MOI 0.05 STARS output.

| | | | Most | | | | | |
|---|---|---|---|---|---|---|---|---|
| Gene | Number of | | enriched | STARS | Average | | | |
| Symbol | spacers | Ranks of spacers | spacer | Score | Score | p-value | FDR | q-value |
| Cd300lf | 4 | 2; 3; 53165; 72390 | 2 | 8.069912369 | 6.033995903 | 0 | 0 | 0 |
| Kmt2d | 4 | 7; 8; 46685; 73018 | 2 | 7.21802944 | 5.336061317 | 0.00034629 | 0.008310969 | 0.008310969 |
| G3bp1 | 4 | 10; 14; 46683; 66311 | 2 | 6.732018789 | 5.015629554 | 0.000779153 | 0.012466453 | 0.012466453 |
| Kdm6a | 4 | 22; 41; 48251; 73037 | 2 | 5.799001719 | 4.378007855 | 0.005367501 | 0.064410008 | 0.064410008 |
| Psmb4 | 4 | 52; 87; 47141; 69239 | 2 | 5.146033021 | 3.86497868 | 0.017660809 | 0.169543762 | 0.169543762 |
| Rpl29 | 4 | 74; 232; 46922; 69574 | 2 | 4.295680144 | 3.36336815 | 0.088304043 | 0.706432344 | 0.592849104 |
| Rps4x | 4 | 208; 251; 46163; 69326 | 2 | 4.227516521 | 3.105968559 | 0.102761666 | 0.704651422 | 0.592849104 |
| Rps20 | 4 | 102; 261; 44898; 67675 | 2 | 4.193692364 | 3.24291932 | 0.110726344 | 0.664358064 | 0.592849104 |
| Sdhd | 4 | 114; 262; 49048; 63810 | 2 | 4.190381738 | 3.217209957 | 0.111159207 | 0.592849104 | 0.592849104 |
| Rpl7a | 4 | 199; 309; 47139; 69262 | 2 | 4.047581803 | 3.025532523 | 0.154791793 | 0.743000606 | 0.723054281 |
| Cflar | 4 | 104; 323; 47337; 68159 | 2 | 4.009247013 | 3.146496441 | 0.165699939 | 0.723054281 | 0.723054281 |
| Rps8 | 4 | 239; 350; 46649; 69131 | 2 | 3.939811689 | 2.932203145 | 0.194961475 | 0.779845901 | 0.75075751 |
| Rpl34 | 4 | 123; 358; 46263; 69398 | 2 | 3.920269364 | 3.065727367 | 0.205177041 | 0.757576767 | 0.75075751 |
| Gapdh | 4 | 154; 373; 46619; 67615 | 2 | 3.884782103 | 2.999429945 | 0.223357285 | 0.765796406 | 0.75075751 |
| Rps24 | 4 | 215; 383; 46315; 69190 | 2 | 3.861911802 | 2.916036047 | 0.234611722 | 0.75075751 | 0.75075751 |
| Rrm2 | 4 | 198; 421; 42207; 67582 | 2 | 3.7801617 | 2.892908214 | 0.280928058 | 0.842784175 | 0.80754913 |
| Nxf1 | 4 | 35; 435; 47124; 69554 | 2 | 3.751900891 | 3.253741133 | 0.297896286 | 0.841118925 | 0.80754913 |
| Hnrnpa3 | 4 | 233; 451; 46807; 61995 | 2 | 3.720701798 | 2.828119965 | 0.320838023 | 0.85556806 | 0.80754913 |
| Eif5a | 4 | 454; 465; 46341; 71490 | 2 | 3.694302547 | 2.671885926 | 0.341182582 | 0.861934943 | 0.80754913 |
| Tnfrsf1a | 4 | 416; 470; 56820; 68572 | 2 | 3.685067591 | 2.685937172 | 0.347588953 | 0.834213488 | 0.80754913 |
| Cdc123 | 4 | 191; 475; 45492; 69342 | 2 | 3.675930945 | 2.84855135 | 0.353302744 | 0.80754913 | 0.80754913 |
| Chic1 | 4 | 480; 546; 51732; 72303 | 2 | 3.555712222 | 2.590711998 | 0.465933685 | 1 | 0.922130974 |
| Eef2 | 4 | 330; 561; 46208; 69394 | 2 | 3.532336524 | 2.659154567 | 0.489221712 | 1 | 0.922130974 |
| Polr2i | 4 | 468; 576; 46184; 67540 | 2 | 3.50958205 | 2.57304586 | 0.519522119 | 1 | 0.922130974 |
| Rps15a | 4 | 388; 582; 46805; 61382 | 2 | 3.500646965 | 2.608627467 | 0.528439096 | 1 | 0.922130974 |
| Cnot3 | 4 | 571; 599; 47258; 67719 | 2 | 3.475826082 | 2.513820792 | 0.558393213 | 1 | 0.922130974 |
| Prpf19 | 4 | 510; 604; 45111; 61050 | 2 | 3.468660796 | 2.534268742 | 0.570080513 | 1 | 0.922130974 |
| Rps29 | 4 | 197; 607; 45221; 69319 | 2 | 3.46439026 | 2.736113776 | 0.576313739 | 0.98796641 | 0.922130974 |
| Psma2 | 4 | 521; 631; 48524; 69355 | 2 | 3.430972726 | 2.510881497 | 0.622976366 | 1 | 0.922130974 |
| Dna2 | 4 | 436; 639; 47399; 78732 | 2 | 3.42011768 | 2.543430111 | 0.637520561 | 1 | 0.922130974 |
| Rpl13a | 4 | 593; 646; 46335; 69616 | 2 | 3.410731325 | 2.4732454 | 0.651545321 | 1 | 0.922130974 |
| Rpl32 | 4 | 596; 652; 47161; 68457 | 2 | 3.402767145 | 2.46819225 | 0.664098346 | 0.99614752 | 0.922130974 |
| Prc1 | 4 | 643; 689; 51877; 69284 | 2 | 3.355230852 | 2.428329208 | 0.741840533 | 1 | 0.922130974 |
| Pdcd2 | 4 | 460; 690; 47420; 68032 | 2 | 3.353982116 | 2.498924082 | 0.744351138 | 1 | 0.922130974 |
| Map3k7 | 4 | 570; 691; 45196; 60179 | 2 | 3.352735204 | 2.452647741 | 0.746169163 | 1 | 0.922130974 |
| Thoc3 | 4 | 417; 692; 47028; 61487 | 2 | 3.351490112 | 2.518635293 | 0.748679768 | 0.998239691 | 0.922130974 |
| Vmn1r5 | 4 | 219; 696; 59047; 66089 | 2 | 3.34652783 | 2.654374042 | 0.757510172 | 0.982715899 | 0.922130974 |
| Ube2n | 4 | 428; 701; 47819; 66687 | 2 | 3.340365288 | 2.507509481 | 0.765821141 | 0.96735302 | 0.922130974 |
| Sf3a2 | 4 | 236; 702; 47579; 68332 | 2 | 3.339138103 | 2.634584688 | 0.768245174 | 0.945532521 | 0.922130974 |
| Gm5082 | 4 | 217; 708; 47315; 60356 | 2 | 3.331811834 | 2.64899183 | 0.783135659 | 0.939762791 | 0.922130974 |
| Srsf1 | 4 | 103; 711; 46233; 67790 | 2 | 3.328172161 | 2.808048882 | 0.789022595 | 0.92373377 | 0.922130974 |
| Dtymk | 4 | 661; 724; 46582; 67642 | 2 | 3.312577301 | 2.40115564 | 0.818543849 | 0.935478685 | 0.922130974 |
| Ddx10 | 4 | 194; 727; 46778; 67805 | 2 | 3.309018631 | 2.66173561 | 0.826075664 | 0.922130974 | 0.922130974 |
| Shq1 | 4 | 132; 735; 46836; 68321 | 2 | 3.299600833 | 2.740132428 | 0.846073933 | 0.922989745 | 0.922989745 |
| Dhps | 4 | 741; 756; 46909; 69261 | 2 | 3.275363117 | 2.358400363 | 0.895506883 | 0.955207341 | 0.955207341 |
| Lars | 4 | 156; 769; 47892; 68101 | 2 | 3.260697181 | 2.684601937 | 0.927105878 | 0.96741483 | 0.96741483 |
| Samm50 | 4 | 610; 785; 53736; 61578 | 2 | 3.242986767 | 2.383375664 | 0.963120076 | 0.983611993 | 0.979222578 |
| Rps17 | 4 | 100; 792; 48594; 67803 | 2 | 3.235352825 | 2.768033256 | 0.979222578 | 0.979222578 | 0.979222578 |

p-value = 0 means <8.65725911177e−05,
FDR = 0 means <0.00415548437365

TABLE 3
MNoV^{CR6} MOI 5 STARS output.

| | | | Most | | | | | |
|---|---|---|---|---|---|---|---|---|
| Gene | Number of | | enriched | STARS | Average | | | |
| Symbol | spacers | Ranks of spacers | spacer | Score | Score | p-value | FDR | q-value |
| Kdm6a | 4 | 105; 193; 37820; 79158 | 2 | 4.455115012 | 3.367360651 | 0.060947104 | 0.548523937 | 0.231408536 |
| Cd300lf | 4 | 1; 209; 3051; 75199 | 2 | 4.386112 | 4.342602536 | 0.071249242 | 0.320621591 | 0.231408536 |
| Ppil1 | 4 | 159; 218; 12364; 37193 | 2 | 4.349590006 | 3.224936674 | 0.077136179 | 0.231408536 | 0.231408536 |
| Kmt2d | 4 | 206; 283; 75202; 79072 | 2 | 4.123641269 | 3.056112588 | 0.129252879 | 0.290818977 | 0.242446541 |
| Hmga1- | 4 | 255; 302; 67191; 72565 | 2 | 4.067408237 | 2.982061562 | 0.147433123 | 0.265379621 | 0.242446541 |
| rs1 | | | | | | | | |
| Atn1 | 4 | 124; 318; 4651; 74196 | 2 | 4.022743066 | 3.115214124 | 0.161631028 | 0.242446541 | 0.242446541 |
| Tmc3 | 4 | 74; 354; 35614; 63453 | 2 | 3.929985072 | 3.180520614 | 0.200848411 | 0.258233672 | 0.258233672 |
| Spp2 | 4 | 50; 666; 33240; 78253 | 2 | 3.38446784 | 2.992696389 | 0.69396589 | 0.780711627 | 0.780711627 |
| Calml4 | 4 | 178; 785; 66901; 78070 | 2 | 3.242986767 | 2.64727936 | 0.963120076 | 0.963120076 | 0.963120076 |

p-value = 0 means <8.65725911177e−05,
FDR = 0 means <0.00415548437365

TABLE 4
MNoV^{CR6} MOI 0.005 STARS output.

| | | | Most | | | | | |
|---|---|---|---|---|---|---|---|---|
| Gene | Number of | | enriched | STARS | Average | | | |
| Symbol | spacers | Ranks of spacers | spacer | Score | Score | p-value | FDR | q-value |
| Cd300lf | 4 | 3; 9; 53165; 72390 | 2 | 7.115735302 | 5.46886992 | 0.00034629 | 0.016621937 | 0.016621937 |
| G3bp1 | 4 | 2; 21; 46683; 66311 | 2 | 6.379912632 | 5.188996034 | 0.001385161 | 0.033243875 | 0.026318068 |
| Smarce1 | 4 | 19; 23; 46671; 73016 | 2 | 6.300917368 | 4.660775673 | 0.001644879 | 0.026318068 | 0.026318068 |
| Kmt2d | 4 | 5; 27; 46685; 73018 | 2 | 6.161689151 | 4.88093883 | 0.002856896 | 0.034282746 | 0.034282746 |
| Sdhd | 4 | 98; 227; 49048; 63810 | 2 | 4.31454971 | 3.312002281 | 0.084408276 | 0.810319453 | 0.587481603 |
| Rps20 | 4 | 107; 228; 44898; 67675 | 2 | 4.310742668 | 3.291093621 | 0.085447147 | 0.683577179 | 0.587481603 |
| Rps4x | 4 | 201; 238; 46163; 69326 | 2 | 4.273567831 | 3.136370447 | 0.093931261 | 0.644100078 | 0.587481603 |
| Rpl29 | 4 | 102; 245; 46922; 69574 | 2 | 4.248466153 | 3.270306214 | 0.097913601 | 0.587481603 | 0.587481603 |
| Psmb4 | 4 | 53; 272; 47141; 69239 | 2 | 4.157955943 | 3.366812063 | 0.119556748 | 0.637635991 | 0.637635991 |
| Rpl7a | 4 | 193; 305; 47139; 69262 | 2 | 4.058855289 | 3.03776795 | 0.150722881 | 0.723469829 | 0.707565657 |
| Cflar | 4 | 109; 319; 47337; 68159 | 2 | 4.02002689 | 3.141730754 | 0.162150463 | 0.707565657 | 0.707565657 |
| Rps8 | 4 | 252; 335; 46649; 69131 | 2 | 3.977693862 | 2.939749577 | 0.179378409 | 0.717513635 | 0.717513635 |
| Rpl34 | 4 | 164; 355; 46263; 69398 | 2 | 3.927545845 | 3.007232214 | 0.201973855 | 0.745749619 | 0.740507315 |
| Gapdh | 4 | 133; 370; 46619; 67615 | 2 | 3.891763446 | 3.034583072 | 0.219634664 | 0.753033133 | 0.740507315 |
| Rps24 | 4 | 187; 380; 46315; 69190 | 2 | 3.86870928 | 2.949503601 | 0.231408536 | 0.740507315 | 0.740507315 |
| Rrm2 | 4 | 171; 419; 42207; 67582 | 2 | 3.784275917 | 2.926578497 | 0.277032292 | 0.831096875 | 0.831096875 |
| Hnrnpa3 | 4 | 246; 446; 46807; 61995 | 2 | 3.730330324 | 2.821251292 | 0.313912215 | 0.886340373 | 0.843147779 |
| Eif5a | 4 | 442; 464; 46341; 71490 | 2 | 3.696161522 | 2.678533493 | 0.340316856 | 0.907511615 | 0.843147779 |
| Tnfrsf1a | 4 | 403; 469; 56820; 68572 | 2 | 3.68690665 | 2.693643958 | 0.346290364 | 0.874838816 | 0.843147779 |
| Cdc123 | 4 | 63; 473; 45492; 69342 | 2 | 3.679573939 | 3.090170578 | 0.351311575 | 0.843147779 | 0.843147779 |
| Chic1 | 4 | 478; 548; 51732; 72303 | 2 | 3.552558354 | 2.590025276 | 0.468963726 | 1 | 0.93022038 |
| Eef2 | 4 | 326; 553; 46208; 69394 | 2 | 3.544724119 | 2.66796368 | 0.476495542 | 1 | 0.93022038 |
| Polr2i | 4 | 467; 573; 46184; 67540 | 2 | 3.514084817 | 2.575753501 | 0.513375465 | 1 | 0.93022038 |
| Rps15a | 4 | 385; 575; 46805; 61382 | 2 | 3.511080342 | 2.615504995 | 0.517357805 | 1 | 0.93022038 |
| Cnot3 | 4 | 574; 597; 47258; 67719 | 2 | 3.478709087 | 2.514149119 | 0.55562289 | 1 | 0.93022038 |
| Prpf19 | 4 | 491; 603; 45111; 61050 | 2 | 3.470089059 | 2.543070818 | 0.568175916 | 1 | 0.93022038 |
| Rps29 | 4 | 173; 606; 45221; 69319 | 2 | 3.465811404 | 2.764837639 | 0.574235997 | 1 | 0.93022038 |
| Psma2 | 4 | 523; 632; 48524; 69355 | 2 | 3.429608281 | 2.509383759 | 0.624534672 | 1 | 0.93022038 |
| Rpl32 | 4 | 595; 635; 47161; 68457 | 2 | 3.425527968 | 2.479929068 | 0.630508181 | 1 | 0.93022038 |
| Rpl13a | 4 | 592; 640; 46335; 69616 | 2 | 3.418770443 | 2.477623211 | 0.640464029 | 1 | 0.93022038 |
| Dna2 | 4 | 435; 645; 47399; 78732 | 2 | 3.412065936 | 2.539894631 | 0.649640724 | 1 | 0.93022038 |
| Prc1 | 4 | 629; 685; 51877; 69284 | 2 | 3.360244148 | 2.435500582 | 0.734308718 | 1 | 0.93022038 |
| Pdcd2 | 4 | 445; 686; 47420; 68032 | 2 | 3.358988061 | 2.508502568 | 0.735953597 | 1 | 0.93022038 |
| Map3k7 | 4 | 591; 687; 45196; 60179 | 2 | 3.357733819 | 2.447463771 | 0.737598476 | 1 | 0.93022038 |
| Thoc3 | 4 | 409; 688; 47028; 61487 | 2 | 3.356481418 | 2.52527154 | 0.739503073 | 1 | 0.93022038 |
| Vmn1r5 | 4 | 167; 692; 59047; 66089 | 2 | 3.351490112 | 2.715292626 | 0.748679768 | 0.998239691 | 0.93022038 |
| Ube2n | 4 | 427; 694; 47819; 66687 | 2 | 3.349005364 | 2.512329242 | 0.753787551 | 0.977886552 | 0.93022038 |
| Sf3a2 | 4 | 249; 699; 47579; 68332 | 2 | 3.342824966 | 2.624891109 | 0.762704528 | 0.963416246 | 0.93022038 |
| Gm5082 | 4 | 185; 704; 47315; 60356 | 2 | 3.336689015 | 2.68581201 | 0.773612674 | 0.952138676 | 0.93022038 |
| Srsf1 | 4 | 108; 707; 46233; 67790 | 2 | 3.333028518 | 2.800224748 | 0.780798199 | 0.936957839 | 0.93022038 |
| Dtymk | 4 | 658; 714; 46582; 67642 | 2 | 3.324547953 | 2.408104005 | 0.794563241 | 0.93022038 | 0.93022038 |
| Ddx10 | 4 | 66; 723; 46778; 67805 | 2 | 3.313766832 | 2.897189891 | 0.816119816 | 0.932708362 | 0.932708362 |
| Shq1 | 4 | 150; 732; 46836; 68321 | 2 | 3.303120326 | 2.714281006 | 0.840186997 | 0.937883159 | 0.937883159 |
| Dhps | 4 | 733; 752; 46909; 69261 | 2 | 3.279926985 | 2.362973365 | 0.887715349 | 0.968416745 | 0.952991083 |
| Nxf1 | 4 | 434; 755; 47124; 69554 | 2 | 3.276501794 | 2.472604099 | 0.89342914 | 0.952991083 | 0.952991083 |
| Lars | 4 | 131; 766; 47892; 68101 | 2 | 3.264059284 | 2.724004777 | 0.91948749 | 0.959465207 | 0.959465207 |
| Samm50 | 4 | 609; 781; 53736; 61578 | 2 | 3.247379955 | 2.385920286 | 0.95454939 | 0.974858951 | 0.972210198 |
| Rps17 | 4 | 106; 789; 48594; 67803 | 2 | 3.238616135 | 2.75706112 | 0.972210198 | 0.972210198 | 0.972210198 |

p-value = 0 means <8.65725911177e−05,
FDR = 0 means <0.000657951692494

TABLE 5
Analysis of CD300 family and guides used in screen.

| | | | | | | | | | CW3 | CW3 | CR6 | CR6 | SEQ |
|---|---|---|---|---|---|---|---|---|---|---|---|---|---|
| | CD300 | CD300 | CD300 | CD300 | CD300 | CD300 | CD300 | CD300 | MOI | MOI | MOI | MOI | ID |
| sgRNA | Ia | Ib | Ic | Id | Ie | If | Ig | Ih | 5 | 0.05 | 5 | 0.05 | NO |
| TGTCACTGTGAAGATGGTGG | | | | | | X^{A} | | | 13.3^{B} | 8.4 | −1.9 | 7.2 | 28 |
| ACAGGTCCAGAGGAGGTGAG | | | | X | | X | | X | 13.0 | 0.0 | 13.1 | 0.0 | 29 |
| GGATCCAGTCACAGGTCCAG | | | | | | X | | X | 12.5 | 0.0 | 12.6 | 0.0 | 30 |
| GTTACTGTGAACATTGGCCC | | | | | | X | | | 12.1 | 0.0 | 12.0 | 0.0 | 31 |
| CTAGCTACTACTCTGATAAC | | | | | | X | | | 11.0 | 0.0 | 9.8 | 0.0 | 32 |
| CCTGTGATCCAGAAGAGAAA | | | | | | X | | | 9.6 | 9.6 | 9.6 | 9.6 | 33 |
| GGTCGTCCCTGATGGACACA | | | | | | | | X | 7.8 | 12.0 | 11.0 | 12.0 | 34 |
| CTCCACGTAGGTACACCGCA | | | | | | | X | | −5.3 | −5.8 | −5.8 | −5.6 | 35 |
| CGTAGCCAGACACAGAGCCA | | | | | X | | | | −5.5 | −5.9 | −5.9 | −5.8 | 36 |
| TCAGAGAAAGAAACGAAGAG | | X | | | | | | | −5.5 | −5.5 | −5.5 | −5.5 | 37 |
| GCCCTTGCTACTTACCTGGG | | | | X | | | | | −5.5 | 0.0 | −5.5 | 0.0 | 38 |
| GCAGCAAATCCAAATCTCAG | | X | | | | | | | −5.8 | −5.8 | −5.8 | −5.8 | 39 |
| GCTACTTACCTGGGAGGAAA | | | | | | | | X | −5.9 | −5.9 | −5.9 | −5.9 | 40 |
| GAGTGTGTCATGTCGATATG | X | | | | | | | | −6.2 | −6.2 | −6.2 | −6.2 | 41 |
| TACCACGCCACAGGACTCCA | | | | | | | X | | −6.2 | 0.0 | −6.2 | 0.0 | 42 |
| GGAAGAGGATGAGCAGAGCA | | | | | | | | | −6.4 | −6.4 | −6.4 | −6.4 | 43 |
| CTACGCAAATCAGGACCAGG | | | | | | | X | | −6.4 | −6.4 | −6.4 | −6.4 | 44 |
| ACGGAAGTGAGAAAGAAAAG | | | | | X | | | | −6.6 | −6.6 | −6.6 | −6.6 | 45 |
| TAGAGCAGAGAACTGCCACA | | | | X | | | | | −6.7 | −6.7 | −6.7 | −6.7 | 46 |
| CATAGGTCACTGTAAAGGTG | X | | | | | | | | −7.2 | −7.2 | −7.2 | −7.2 | 47 |

^{A}An “X” indicates an identical match between the sgRNA and genomic DNA sequence of the corresponding CD300 gene
^{B}The number represents a log₂ fold enrichment after MNV infection relative to mock infected cells.

TABLE 6
Crystallographic data collection and refinement statistics.

| | sCD300lf | |
|---|---|---|
| Space group | P 2₁ 2₁ 2₁ | |
| Cell constants a, b, c (Å) | 27.63, 53.35, 70.52 | |
| Resolution (Å) | 18.52-1.603 | |
| Total reflections | 74844 | |
| Unique reflections | 13664 | (1195) |
| Multiplicity | 5.48 | |
| Completeness (%) | 95.4 | (19.19-1.60) |
| | 90.4 | (1.66-1.60) |
| Mean I/sigma I | 21.56 | (2.11) |
| Wilson B-factor (Å2) | 26.9 | |
| R-merge** | 0.06 | |
| Reflections used in refinement | 13664 | (1195) |

| Reflections used for R-free | 1289 reflections, 9.98% |
|---|---|
| R-work/R-free*** | 0.1608 (0.3118)/0.1977 (0.3692) |
| Number of non-hydrogen atoms | 1035 |
| Protein residues | 113 |
| RMS (bonds) | 0.008 |
| RMS (angles) | 1.14 |
| Ramachandran favored (%) | 100 |
| Rotamer outliers (%) | 0.99 |
| Average B-factors | 34.43 |
| Protein | 33.20 |
| HEPES/sodium | 37.47/30.6 |
| Solvent | 40.74 |

*Statistics for the highest-resolution shell are shown in parentheses.
**Rmerge = Σhkl Σi lli(hkl) − lhkll/Σhkl Σi l(hkl).
***Rwork and Rfree = Σ||Fo| − |Fc||/Σ|Fo|, where Fo and Fc are the observed and calculated structure factor amplitudes. For the calculation of Rfree, 10% of the reflection data were selected and omitted from refinement

TABLE 7

| SEQ | | |
|---|---|---|
| ID | | |
| NO: | Description | SEQUENCE |
| 1 | CD300If- | GTGCAGTGCCGATATACCTC |
| | specific | |
| | sgRNA | |
| 2 | CD300Id- | AGATATTCCTCATACTGGAA |
| | specific | |
| | sgRNA | |
| 3 | CD300If | AGGATGTCGAGGGATGGCAGGCAGC |
| | forward | |
| | primer | |
| 4 | CD300If | TGCCAGCATCGCTCATCCTCAGATCC |
| | reverse | |
| | primer | |
| 5 | CD300Id | CACCGAGATATTCCTCATACTGGAA |
| | forward | |
| | primer | |
| 6 | CD300Id | AAACTTCCAGTATGAGGAATATCTC |
| | reverse | |
| | primer | |
| 7 | CD300If aa | QEQGSLTVQCRYTSGWKDYK |
| | 13-32 | |
| 8 | CD300If nt | CAGGAGCAGGGCTCCTTGACAGTGCAGTGCCGATATACCTCAGGCTGGAAGGATTACA |
| | fragment | AG |
| 9 | CD300If nt | CAGGAGCAGGGCTCCTTGACAGTGCAGTGCCGATATCTCAGGCTGGAAGGATTACAAG |
| | fragment | |
| | clone 1.1 | |
| 10 | CD300If nt | CAGGAGCAGGGCTCCTTGACAGTGCAGTGCCGATATACTCAGGCTGGAAGGATTACAA |
| | fragment | G |
| | clone 1.2 | |
| 11 | CD300If nt | CAGGAGCAGGGAG |
| | fragment | |
| | clone 1.3 | |
| 12 | CD300If nt | CAGGAGCAGGGCTCCTTGACAGTGCAGTGCCCTCAGGCTGGAAGGATTACAAG |
| | fragment | |
| | clone 2.1 | |
| 13 | CD300If nt | CAGGAGCAGGGCTCCTTGACAGTGCAGTGCCGATATACTCAGGCTGGAAGGATTACAA |
| | fragment | G |
| | clone 2.2 | |
| 14 | CD300Id aa | EQGSLTVQCRYSSYWKGYKK |
| | fragment | |
| 15 | CD300Id nt | GAGCAGGGCTCCTTGACAGTGCAGTGCAGATATTCCTCATACTGGAAGGGTTACAAGAA |
| | fragment | G |
| 16 | CD300Id nt | GAGCAGGGCTCCTTGACAGTGCAGTGCAGATATTCCTCAGGGTTACAAGAAG |
| | fragment | |
| | clone 1.1 | |
| 17 | CD300Id nt | GAGCAGGGCTCCTTGACAGTGCAGTGCAGATATTCCTCATACTGAAGGGTTACAAGAAG |
| | fragment | |
| | clone 1.2 | |
| 18 | CD300Id nt | GAGCAGGGCTCCTTGACAGTGCAGTGCAGATATTCCTCATACTGAAGGGTTACAAGAAG |
| | fragment | |
| | clone 2.1 | |
| 19 | CD300Id nt | GAGCAGGGCTCCTTGACAGTGCAGTGCAGATATTCCTCATACGGAAGGGTTACAAGAAG |
| | fragment | |
| | clone 2.2 | |
| 20 | qPCR | CACGCCACCGATCTGTTCTG |
| | forward | |
| | primer | |
| 21 | qPCR | GCGCTGCGCCATCACTC |
| | reverse | |
| | primer | |
| 22 | mCD300If | KDYK |
| | CDR1 aa | |
| | 29-32 | |
| 23 | hCD300If | ETYL |
| | CDR1 aa | |
| | 29-32 | |
| 24 | mCD300If | RDFI |
| | ED loop aa | |
| | 69-72 | |
| 25 | hCD300If | KNRT |
| | ED loop aa | |
| | 69-72 | |
| 26 | mCD300If | TKGGLDPMFK |
| | CDR3 aa | |
| | 93-102 | |
| 27 | hCD300If | EKTGNDLGVT |
| | CDR3 aa | |
| | 23-102 | |
| 28 | sgRNA1 | TGTCACTGTGAAGATGGTGG |
| 29 | sgRNA2 | ACAGGTCCAGAGGAGGTGAG |
| 30 | sgRNA3 | GGATCCAGTCACAGGTCCAG |
| 31 | sgRNA4 | GTTACTGTGAACATTGGCCC |
| 32 | sgRNA5 | CTAGCTACTACTCTGATAAC |
| 33 | sgRNA6 | CCTGTGATCCAGAAGAGAAA |
| 34 | sgRNA7 | GGTCGTCCCTGATGGACACA |
| 35 | sgRNA8 | CTCCACGTAGGTACACCGCA |
| 36 | sgRNA9 | CGTAGCCAGACACAGAGCCA |
| 37 | sgRNA10 | TCAGAGAAAGAAACGAAGAG |
| 38 | sgRNA11 | GCCCTTGCTACTTACCTGGG |
| 39 | sgRNA12 | GCAGCAAATCCAAATCTCAG |
| 40 | sgRNA13 | GCTACTTACCTGGGAGGAAA |
| 41 | sgRNA14 | GAGTGTGTCATGTCGATATG |
| 42 | sgRNA15 | TACCACGCCACAGGACTCCA |
| 43 | sgRNA16 | GGAAGAGGATGAGCAGAGCA |
| 44 | sgRNA17 | CTACGCAAATCAGGACCAGG |
| 45 | sgRNA18 | ACGGAAGTGAGAAAGAAAAG |
| 46 | sgRNA19 | TAGAGCAGAGAACTGCCACA |
| 47 | sgRNA20 | CATAGGTCACTGTAAAGGTG |
| 48 | mCD300If | EDPVTGPEEVSGQEQGSLTVQCRYTSGWKDYKKYWCQGVPQRSCKTLVETDASEQLVKK |
| | | NRVSIRDNQRDFIFTVTMEDLRMSDAGIYWCGITKGGLDPMFKVTVNIGPA |
| 49 | mCD300Id | QDSVTGPEEVSGQEQGSLTVQCRYSSYWKGYKKYVVCRGVPQRSCDILVETDKSEQLVKK |
| | | NRVSIRDNQRDlFIFTVTMEDLRMSDAGIYWCGITKGGPDPMFKVNVNIDQA |
| 50 | mCD300Ih | QDPVTGPEEVSGQEQGSLTVQCRYDSGWKDYKKYVVCRGAYWKSCEILVETDASEQLVKE |
| | | NRVSIRDDQTDFIFTVTMEDLRMSDADIYVVCGITKAGTDPMFKVNVNIDPE |
| 51 | mCD300Ib | IQGPALVRGPEQGSVTVQCRYSSRWQTNKKWWCRGASWSTCRVLIRSTGSEKETKSGRL |
| | | SIRDNQKNHSFQVTMEMLRQNDTDTYVVCGIEKFGTDRGTRVKVNVYSV |
| 52 | mCD300e | LTGPGSVSGYVGGSLRVQCQYSPSYKGYMKYWCRGPDHTTCKTIVETDGSEKEKRSGPVS |
| | | IRDHASNSTITVIMEDLSEDNAGSYVVCKIQTSFIWDSWSRDPSVSVRVNVFPA |
| 53 | mCD300c | HFPVRGPSTVTGTVGESLSVSCQYEKKLKTKKKIWCKWKSNVLCKDIVKTSASEEARNRGV |
| | | SIRDHPDNLTFTVTLENLTLEDDAGTYMCMDIGFFYDAYLQIDKSFKVEVFVVPG |
| 54 | mCD300a | LHGPSTMSGSVGESLSVSCRYEEKFKTKDKYWCRVSLKILCKDIVKTSSSEEARSGRVTIRD |
| | | HPDNLTFTVTYESLTLEDADTYMCAVDISLFDGSLGFDKYFKIELSVVPS |
| 55 | mCD300Ig | LKGPKEISGFEGDTVSLRCTYVEKMKEHRKYVVCRQGGILVSRCGDIVYANQDQEVTRGRM |
| | | SIRDSPQELSMTVIMRDLTLKDSGKYWCGIDRLGRDESFEVTLIVFPG |
| 56 | hCD300If | QITGPTTVNGLERGSLTVQCVYRSGWETYLKWWCRGAIWRDCKILVKTSGSEQEVKRDRV |
| | | SIKDNQKNRTFTVTMEDLMKTDADTYVVCGIEKTGNDLGVTVQVTIDPA |
| 57 | CD300If | FLMHLSLLVPFLFWITGCCTAEDPVTGPEEVSGQEQGSLTVQCRYTSGWKDYKKYVVCQGVPQ |
| | | RSCKTLVETDASEQLVKKNRVSIRDNQRDFIFTVTMEDLRMSDAGIYVVCGITKGGLDPMFKV |
| | | TVNIGPAIQVPITVPTMPPITSTTTIFTVTTTVKETSMFPTLTSYYSDNGHGGGDSGGGEDGV |
| | | GDGFLDLSVLLPVISAVLLLLLLVASLFAWRMVRRQKKAAGPPSEQAQSLEGDLCYADLSLK |
| | | QPRTSPGSSWKKGSSMSSSGKDHQEEVEYVTMAPFPREEVSYAALTLAGLGQEPTYGNTG |
| | | CPITHVPRTGLEEETTEYSSIRRPLPAAMP |
| 58 | CD300Id | FLMWQFSALLLFFLPGCCTAQDSVTGPEEVSGQEQGSLTVQCRYSSYWKGYKKYVVCRGVPQ |
| | | RSCDILVETDKSEQLVKKNRVSIRDNQRDFIFTVTMEDLRMSDAGIYVVCGITKGGPDPMFKV |
| | | NVNIDQAPKSSMMTTTATVLKSIQPSAENTGKEQVTQSKEVTQSRPHTRSLLSSIYFLLMVFV |
| | | ELPLLLSMLSAVLWVTRPQRCFGRGENDLVKTHSPVA |

### REFERENCES FOR THE EXAMPLE

1. Patel, M. M., et al., Systematic literature review of role of noroviruses in sporadic gastroenteritis. Emerg. Infect. Dis., 2008. 14(8): p. 1224-1231.
2. Scallan, E., et al., Foodborne illness acquired in the United States-major pathogens. Emerg Infect Dis, 2011. 17(1): p. 7-15.
3. Jones, M. K., et al., Enteric bacteria promote human and murine norovirus infection of B cells. Science, 2014. 346(6210): p. 755-9.
4. Kernbauer, E., Y. Ding, and K. Cadwell, An enteric viral infection can functionally replace the beneficial cues provided by commensal bacteria. Nature, 2014. 516(7529): p. 94-98.
5. Karst, S. M., et al., Advances in norovirus biology. Cell Host Microbe, 2014. 15(6): p. 668-80.
6. Baldridge, M. T., et al., Commensal microbes and interferon-lambda determine persistence of enteric murine norovirus infection. Science, 2015. 347(6219): p. 266-9.
7. Nice, T. J., et al., Interferon-lambda cures persistent murine norovirus infection in the absence of adaptive immunity. Science, 2015. 347(6219): p. 269-73.
8. Newman, K. L. and J. S. Leon, Norovirus immunology: Of mice and mechanisms. Eur J Immunol, 2015. 45(10): p. 2742-57.
9. Taube, S., et al., A mouse model for human norovirus. MBio, 2013. 4(4).
10. Hutson, A. M., R. L. Atmar, and M. K. Estes, Norovirus disease: changing epidemiology and host susceptibility factors. Trends in Microbiology, 2004. 12(6): p. 279-287.
11. Hutson, A. M., et al., Norwalk virus infection and disease is associated with ABO histo-blood group type. J. Infect. Dis., 2002. 185(9): p. 1335-1337.
12. Taube, S., et al., Murine noroviruses bind glycolipid and glycoprotein attachment receptors in a strain-dependent manner. J Virol, 2012. 86(10): p. 5584-93.
13. Ossiboff, R. J., et al., Conformational changes in the capsid of a calicivirus upon interaction with its functional receptor. J Virol, 2010. 84(11): p. 5550-64.
14. Ossiboff, R. J. and J. S. Parker, Identification of regions and residues in feline junctional adhesion molecule required for feline calicivirus binding and infection. J. Virol., 2007. 81(24): p. 13608-13621.
15. Strong, D. W., et al., Protruding domain of capsid protein is necessary and sufficient to determine murine norovirus replication and pathogenesis in vivo. J Virol, 2012. 86(6): p. 2950-8.
16. Wobus, C. E., et al., Replication of Norovirus in cell culture reveals a tropism for dendritic cells and macrophages. PLoS Biol, 2004. 2(12): p. e432.
17. Ward, V. K., et al., Recovery of infectious murine norovirus using pol II-driven expression of full-length cDNA. Proc Natl Acad Sci USA, 2007. 104(26): p. 11050-5.
18. Marquez, J. A., et al., The crystal structure of the extracellular domain of the inhibitor receptor expressed on myeloid cells IREM-1. J Mol Biol, 2007. 367(2): p. 310-8.
19. Lindesmith, L., et al., Human susceptibility and resistance to Norwalk virus infection. Nat. Med, 2003. 9(5): p. 548-553.
20. Kambhampati, A., et al., Host Genetic Susceptibility to Enteric Viruses: A Systematic Review and Metaanalysis. Clin Infect Dis, 2015.

### REFERENCES FOR THE METHODS

1. Doench, J. G., et al., Rational design of highly active sgRNAs for CRISPR-Cas9-mediated gene inactivation. Nat Biotechnol, 2014.
2. Doench, J. G., et al., Optimized sgRNA design to maximize activity and minimize off-target effects of CRISPR-Cas9. Nature Biotechnology, 2015.
2. Strong, D. W., et al., Protruding domain of capsid protein is necessary and sufficient to determine murine norovirus replication and pathogenesis in vivo. J Virol, 2012. 86(6): p. 2950-8.
3. Nybakken, G. E., et al., Crystal structure of the West Nile virus envelope glycoprotein. J Virol, 2006. 80: p. 11467-11474.
4. Baert, L., et al., Detection of murine norovirus 1 by using plaque assay, transfection assay, and real-time reverse transcription-PCR before and after heat exposure. Appl Environ Microbiol, 2008. 74(2): p. 543-546.
5. Wang, G. G., et al., Quantitative production of macrophages or neutrophils ex vivo using conditional Hoxb8. Nat Methods, 2006. 3(4): p. 287-93.
6. Takeshita, S., K. Kaji, and A. Kudo, Identification and Characterization of the New Osteoclast Progenitor with Macrophage Phenotypes Being Able to Differentiate into Mature Osteoclasts. Journal of Bone and Mineral Research, 2000. 15(8): p. 1477-1488.
7. Durbin, J. E., et al., Targeted disruption of the mouse Stat1 gene results in compromised innate immunity to viral disease. Cell, 1996. 84(3): p. 443-450.
8. Durbin, J. E., et al., Targeted disruption of the mouse Stat1 gene results in compromised innate immunity to viral disease. Cell, 1996. 84(3): p. 443-450.
9. Otwinowski, Z. and W. Minor, Processing of X-ray diffraction data collected in oscillation mode. Macromolecular Crystallography, Pt A, 1997. 276: p. 307-326.
10. McCoy, A. J., et al., Phaser crystallographic software. J Appl Crystallogr, 2007. 40(Pt 4): p. 658-674.
11. Adams, P. D., et al., PHENIX: a comprehensive Python-based system for macromolecular structure solution. Acta Crystallogr D Biol Crystallogr, 2010. 66(Pt 2): p. 213-21.
12. Emsley, P., et al., Features and development of Coot. Acta Crystallogr D Biol Crystallogr, 2010. 66(Pt 4): p. 486-501.
13. Tian, L., et al., p85alpha recruitment by the CD300f phosphatidylserine receptor mediates apoptotic cell clearance required for autoimmunity suppression. Nat Commun, 2014. 5: p. 3146.
14. Zheng, H., et al., Validation of metal-binding sites in macromolecular structures with the CheckMyMetal web server. Nat Protoc, 2014. 9(1): p. 156-70.

What is claimed is:

1. A mammalian cell, wherein the cell expresses at least a portion of a heterologous CD300lf or CD300ld protein.

2. The cell of claim 1, wherein the cell is susceptible to infection by murine norovirus (MNoV).

3. The cell of claim 1, wherein the cell expresses the ectodomain of CD300lf or CD300ld.

4. The cell of claim 1, wherein the heterologous CD300lf comprises the amino acid sequence of SEQ ID NO: 57 or the heterologous CD300ld comprises the amino acid sequence of 58.

5. The cell of claim 1, wherein the cell comprises a nucleic acid encoding at least a portion of a heterologous CD300lf or CD300ld protein.

6. The cell of claim 1, wherein the portion of CD300lf comprises the amino acid sequence of SEQ ID NO: 48 or the portion of CD300ld comprises the amino acid sequence of SE ID NO: 49.

7. The cell of claim 1, wherein the cell is a human cell.

8. A method of screening a test compound for antiviral activity to treat or prevent a norovirus infection, the method comprising: (a) contacting the cell of claim 1 with a test compound and MNoV; and (b) determining MNoV titers, wherein an amount of MNoV that is reduced relative to control indicates an antiviral compound that may treat a norovirus infection.

9. The method of claim 8, wherein the control is not contacted with the test compound.

10. The method of claim 8, wherein an amount of MNoV that is reduced relative to control indicates an antiviral compound that may treat a MNoV infection.

11. The method of claim 8, wherein an amount of MNoV that is reduced relative to control indicates an antiviral compound that may treat a human norovirus (HNoV) infection.

12. A composition for treating or preventing a norovirus infection comprising a therapeutically effective amount of at least a portion of CD300lf or CD300ld and a pharmaceutically acceptable excipient.

13. The composition of claim 12, wherein the portion of CD300lf or CD300ld is a soluble ectodomain.

14. The composition of claim 13, wherein the portion of CD300lf comprises the amino acid sequence of SEQ ID NO: 48 or the portion of CD300ld comprises the amino acid sequence of SEQ ID NO: 49.

15. The composition of claim 12, wherein the norovirus is MNoV.

16. A method to treat a norovirus infection in a subject, the method comprising administering to the subject a therapeutically effective amount of a composition comprising at least a portion of CD300lf or CD300ld.

17. The method of claim 17, wherein the norovirus is a MNoV.

18. The method of claim 17, wherein the portion of CD300lf or CD300ld is a soluble ectodomain.

19. The method of claim 17, wherein the portion of CD300lf comprises the amino acid sequence of SEQ ID NO: 48 or the portion of CD300ld comprises the amino acid sequence of SEQ ID NO: 49.
